(19) United States
(12) Patent Application Publication
(10) Pub. No.: US 2025/0042872 A1
(43) Pub. Date: Feb. 6, 2025

(54) NOVEL SALT OF 1-SULFONYL PYRROLE DERIVATIVE, METHOD FOR PREPARING SAME, AND PHARMACEUTICAL COMPOSITION INCLUDING SAME

(71) Applicant: ILDONG PHARMACEUTICAL CO., LTD., Seoul (KR)

(72) Inventors: Hong Chul YOON, Hwaseong-si (KR); Joon Tae PARK, Hwaseong-si (KR); Jung Woo LEE, Hwaseong-si (KR); Kyung Mi AN, Hwaseong-si (KR); Chang Hee HONG, Hwaseong-si (KR); Jae Eui SHIN, Hwaseong-si (KR); Soo Jin LEE, Hwaseong-si (KR); Hanna SEO, Hwaseong-si (KR); Jae Hong LEE, Hwaseong-si (KR)

(21) Appl. No.: 18/719,710

(22) PCT Filed: Dec. 14, 2022
(86) PCT No.: PCT/KR2022/020380

§ 371 Date: Jun. 13, 2024

(30) Foreign Application Priority Data
Dec. 15, 2021 (KR) 1020210180072

(51) Int. Cl. C07D 401/12

(52) U.S. Cl. CPC C07D 401/12

(57) ABSTRACT

The present disclosure relates to a novel salt of a 1-sulfonyl pyrrole derivative, and to a novel salt having excellent solubility in vivo, stability, bioavailability, and the like, a preparation method thereof, and a pharmaceutical composition comprising the same.

### TECHNICAL FIELD

The present disclosure relates to a novel salt of 1-sulfonyl pyrrole derivative, which is difficult to be prepared by a conventional salt preparation process, a preparation method thereof, and a pharmaceutical composition comprising the same.

### BACKGROUND ART

Potassium competitive acid blockers (P-CABs)-based anti-ulcer drugs inhibit gastric acid secretion by competitively binding to a K⁺ binding site of a proton pump, which is located in the final stage of acid secretion in gastric parietal cells, to interfere with the exchange process of protons (H⁺). The P-CABs are next-generation drugs that compensate for the shortcomings of proton pump inhibitor (PPI) drugs such as omeprazole, esomeprazole, and ilaprazole, which are commonly prescribed in the gastric acid secretion inhibitor market at present.

These potassium-competitive gastric acid secretion inhibitors are in the spotlight as a new class of drugs, but the number of related drugs available on the market is still quite limited.

Meanwhile, many free bases may have problems in some cases in that the free bases exist in the form of oil at room temperature of 15 to 25° C., or it is not easy to handle industrially.

Therefore, there is a need for research on a method for providing a pharmaceutical product that is advantageous in product handling and storage due to superior physical and chemical stability, non-hygroscopicity, and the like, achieves mass-production, and has excellent solubility, resulting in providing improved bioavailability. In particular, in order for a drug to exhibit rapid pharmacological activity in vivo, the drug should rapidly elute from the digestive tract. This is closely related to the drug solubility, that is, the higher the solubility of the drug, the higher the drug dissolution rate and the rate of absorption in the digestive tract, and the higher the rate of absorption in the digestive tract, the faster and more effective blood concentration is achieved even at low doses, and thus it is possible to expect high efficacy and bioavailability of the drug.

Accordingly, it is also required to select the optimal salt form. Under this background, the present inventors repeatedly researched, developed novel salts of 1-(5-(2-fluorophenyl)-4-methoxy-1-((6-methoxypyridin-3-yl)sulfonyl)-1H-pyrrole-3-yl)-N-methylmethanamine, found that these salts had excellent stability, solubility and bioavailability and high purity, and further made a lot of research to mass-produce these salts, and then completed the present disclosure.

### DISCLOSURE

### Technical Problem

An object of the present disclosure is to provide a novel salt of 1-(5-(2-fluorophenyl)-4-methoxy-1-((6-methoxypyridin-3-yl)sulfonyl)-1H-pyrrole-3-yl)-N-methylmethanamine. In particular, another object of the present disclosure is to provide a novel salt with improved physicochemical and/or pharmaceutical properties such as solubility (specifically, solubility in vivo), stability (dissolution stability, storage stability, etc.), and the like, a preparation method thereof, and a pharmaceutical composition comprising the same.

### Technical Solution

In order to achieve the above object, there are provided a novel salt of 1-(5-(2-fluorophenyl)-4-methoxy-1-((6-methoxypyridin-3-yl)sulfonyl)-1H-pyrrole-3-yl)-N-methylmethanamine, a preparation method thereof, and a pharmaceutical composition comprising the same as an active ingredient.

Hereinafter, each detailed description is provided below.

### Novel salt of 1-(5-(2-fluorophenyl)-4-methoxy-1-((6-methoxypyridin-3-yl)sulfonyl)-1H-pyrrol-3-yl)-N-methylmethanamine

In order to achieve the above object, the present disclosure provides a hydrochloride salt of 1-(5-(2-fluorophenyl)-4-methoxy-1-((6-methoxypyridin-3-yl)sulfonyl)-1H-pyrrole-3-yl)-N-methylmethanamine.

The hydrochloride salt of 1-(5-(2-fluorophenyl)-4-methoxy-1-((6-methoxypyridin-3-yl)sulfonyl)-1H-pyrrole-3-yl)-N-methylmethanamine may be represented by the following Chemical Formula I.

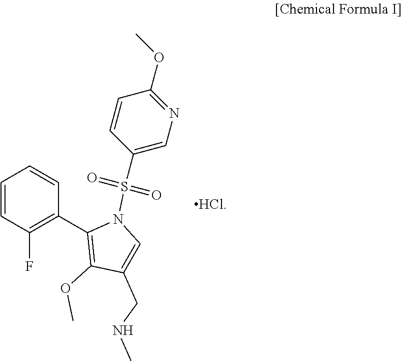 [SMILES: CNCc1cn(S(=O)(=O)c2ccc(OC)nc2)c(-c2ccccc2F)c1OC]

The novel salt according to the present disclosure exhibits excellent physicochemical properties in various aspects, such as stability, solubility in vivo, bioavailability, and the like.

Preferably, the novel salt is a crystalline form.

The crystalline form may be a crystalline form I or a crystalline form II.

The crystalline form I of the hydrochloride salt of the present disclosure may comprise, in an X-ray powder diffraction (XRPD) graph, at least three diffraction peaks at 2-theta (2θ) angle values selected from the group consisting of 16.53±0.2, 20.53±0.2, 21.32±0.2, 24.25±0.2, 26.78±0.2, 27.32±0.2 and 28.04±0.2. More specifically, the crystalline form of the hydrochloride salt of the present disclosure may comprise, in an X-ray powder diffraction (XRPD) graph, diffraction peaks at 2-theta (2θ) angle values of 16.53±0.2, 20.53±0.2, 21.32±0.2, 24.25±0.2, 26.78±0.2, 27.32±0.2 and 28.04±0.2.

Even more specifically, the crystalline form I of the hydrochloride salt of the present disclosure may further comprise, in an X-ray powder diffraction (XRPD) graph, any one or more diffraction peaks at 2-theta (2θ) angle values selected from the group consisting of 8.97±0.2, 18.15±0.2, 25.45±0.2, and 32.40±0.2.

Further, the crystalline form I of the hydrochloride salt of 1-(5-(2-fluorophenyl)-4-methoxy-1-((6-methoxypyridin-3-yl)sulfonyl)-1H-pyrrole-3-yl)-N-methylmethanamine of the present disclosure may exhibit an X-ray powder diffraction spectroscopy pattern of FIG. 1.

The crystalline form I of the hydrochloride salt of the present disclosure is characterized by having an endothermic transition peak value at 177 to 190° C. when a temperature increase rate is 20° C./min in a differential scanning calorimetry (DSC) graph, and preferably, characterized by having an endothermic transition peak value at 179 to 188° C., and more preferably at 183±2° C.

Further, the crystalline form I of the hydrochloride salt of 1-(5-(2-fluorophenyl)-4-methoxy-1-((6-methoxypyridin-3-yl)sulfonyl)-1H-pyrrole-3-yl)-N-methylmethanamine of the present disclosure may show the differential scanning calorimetry of FIG. 2.

In addition, the crystalline form I of the hydrochloride salt of 1-(5-(2-fluorophenyl)-4-methoxy-1-((6-methoxypyridin-3-yl)sulfonyl)-1H-pyrrole-3-yl)-N-methylmethanamine of the present disclosure may have a thermogravimetric analysis (TGA) pattern showing a weight loss of less than 0.2 wt % at 120° C. or less. Specifically, the thermogravimetric analysis (TGA) pattern of FIG. 3 may be shown.

The crystalline form II of the hydrochloride salt of the present disclosure may comprise, in an X-ray powder diffraction (XRPD) graph, at least three diffraction peaks at 2-theta (2θ) angle values selected from the group consisting of 7.96±0.2, 13.22±0.2, 15.64±0.2, 16.04±0.2, 18.16±0.2, 22.88±0.2 and 25.18±0.2. More specifically, the crystalline form of the hydrochloride salt of the present disclosure may comprise, in an X-ray powder diffraction (XRPD) graph, diffraction peaks at 2-theta (2θ) angle values of 7.96±0.2, 13.22±0.2, 15.64±0.2, 16.04±0.2, 18.16±0.2, 22.88±0.2 and 25.18±0.2.

Even more specifically, the crystalline form II of the hydrochloride salt of the present disclosure may further comprise, in an X-ray powder diffraction (XRPD) graph, any one or more diffraction peaks at 2-theta (2θ) angle values selected from the group consisting of 21.12±0.2, 24.12±0.2, 27.80±0.2, and 31.12±0.2.

Further, the crystalline form II of the hydrochloride salt of 1-(5-(2-fluorophenyl)-4-methoxy-1-((6-methoxypyridin-3-yl)sulfonyl)-1H-pyrrole-3-yl)-N-methylmethanamine of the present disclosure may exhibit an X-ray powder diffraction spectroscopy pattern of FIG. 5.

The crystalline form II of the hydrochloride salt of the present disclosure is characterized by having an endothermic transition peak value at 179 to 191° C. when a temperature increase rate is 20° C./min in a differential scanning calorimetry (DSC) graph, and preferably, characterized by having an endothermic transition peak value at 177 to 189° C., and more preferably at 185±2° C.

Further, the crystalline form II of the hydrochloride salt of 1-(5-(2-fluorophenyl)-4-methoxy-1-((6-methoxypyridin-3-yl)sulfonyl)-1H-pyrrole-3-yl)-N-methylmethanamine of the present disclosure may show the differential scanning calorimetry of FIG. 6.

In addition, the crystalline form II of the hydrochloride salt of 1-(5-(2-fluorophenyl)-4-methoxy-1-((6-methoxypyridin-3-yl)sulfonyl)-1H-pyrrole-3-yl)-N-methylmethanamine of the present disclosure may have a thermogravimetric analysis (TGA) pattern showing a weight loss of less than 0.1 wt % at 120° C. or less. Specifically, the thermogravimetric analysis (TGA) pattern of FIG. 7 may be shown.

In order to achieve the above object, the present disclosure provides a succinate salt of 1-(5-(2-fluorophenyl)-4-methoxy-1-((6-methoxypyridin-3-yl)sulfonyl)-1H-pyrrole-3-yl)-N-methylmethanamine.

The succinate salt of 1-(5-(2-fluorophenyl)-4-methoxy-1-((6-methoxypyridin-3-yl)sulfonyl)-1H-pyrrole-3-yl)-N-methylmethanamine may be represented by the following Chemical Formula II.

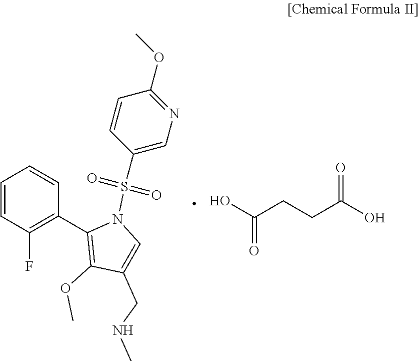 [SMILES: CNCc1cn(S(=O)(=O)c2ccc(OC)nc2)c(-c2ccccc2F)c1OC ; O=C(O)CCC(=O)O]

The novel salt according to the present disclosure exhibits excellent physicochemical properties in various aspects, such as stability, solubility in vivo, bioavailability, and the like.

Preferably, the novel salt is a crystalline form.

The crystalline form of the succinate salt of the present disclosure may comprise, in an X-ray powder diffraction (XRPD) graph, at least three diffraction peaks at 2-theta (2θ) angle values selected from the group consisting of 17.14±0.2, 18.70±0.2, 19.74±0.2, 21.51±0.2, 22.75±0.2, 23.53±0.2, 25.81±0.2, and 28.40±0.2. More specifically, the crystalline form of the succinate salt of the present disclosure may comprise, in an X-ray powder diffraction (XRPD) graph, diffraction peaks at 2-theta (2θ) angle values of 17.14±0.2, 18.70±0.2, 19.74±0.2, 21.51±0.2, 22.75±0.2, 23.53±0.2, 25.81±0.2, and 28.40±0.2.

Even more specifically, the crystalline form of the succinate salt of the present disclosure may further comprise, in an X-ray powder diffraction (XRPD) graph, any one or more diffraction peaks at 2-theta (2θ) angle values selected from the group consisting of 12.75±0.2, 14.09±0.2, 15.02±0.2, 20.42±0.2, and 29.82±0.2.

In addition, the succinate salt of 1-(5-(2-fluorophenyl)-4-methoxy-1-((6-methoxypyridin-3-yl)sulfonyl)-1H-pyrrole-3-yl)-N-methylmethanamine of the present disclosure may exhibit an X-ray powder diffraction spectroscopy pattern of FIG. 11.

The crystalline form of the succinate salt of the present disclosure is characterized by having an endothermic transition peak value at 135 to 147° C. when a temperature increase rate is 20° C./min in a differential scanning calorimetry (DSC) graph, and preferably, characterized by having an endothermic transition peak value at 137 to 145° C., and more preferably at 141±2° C.

In addition, the succinate salt of 1-(5-(2-fluorophenyl)-4-methoxy-1-((6-methoxypyridin-3-yl)sulfonyl)-1H-pyrrole-3-yl)-N-methylmethanamine of the present disclosure may show the differential scanning calorimetry of FIG. 8 or 12.

In addition, the succinate salt of 1-(5-(2-fluorophenyl)-4-methoxy-1-((6-methoxypyridin-3-yl)sulfonyl)-1H-pyrrole-3-yl)-N-methylmethanamine of the present disclosure may have a thermogravimetric analysis (TGA) pattern showing a weight loss of less than 0.2 wt % at 120° C. or less. Specifically, the thermogravimetric analysis (TGA) pattern of FIG. 9 or 13 may be shown.

In order to achieve the above object, the present disclosure provides an L-tartrate salt of 1-(5-(2-fluorophenyl)-4-methoxy-1-((6-methoxypyridin-3-yl)sulfonyl)-1H-pyrrole-3-yl)-N-methylmethanamine.

The L-tartrate salt of 1-(5-(2-fluorophenyl)-4-methoxy-1-((6-methoxypyridin-3-yl)sulfonyl)-1H-pyrrole-3-yl)-N-methylmethanamine may be represented by the following Chemical Formula III.

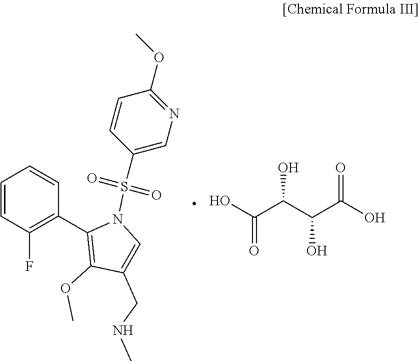 [SMILES: CNCc1cn(S(=O)(=O)c2ccc(OC)nc2)c(-c2ccccc2F)c1OC ; O=C(O)[C@H](O)[C@@H](O)C(=O)O]

The novel salt according to the present disclosure exhibits excellent physicochemical properties in various aspects, such as stability, solubility in vivo, bioavailability, and the like.

Preferably, the novel salt is a crystalline form.

The crystalline form of the L-tartrate salt of the present disclosure may comprise, in an X-ray powder diffraction (XRPD) graph, at least three diffraction peaks at 2-theta (2θ) angle values selected from the group consisting of 8.99±0.2, 13.52±0.2, 17.54±0.2, 18.07±0.2, 20.74±0.2, 21.79±0.2, 22.57±0.2, and 24.68±0.2.

More specifically, the crystalline form of the L-tartrate salt of the present disclosure may comprise, in an X-ray powder diffraction (XRPD) graph, diffraction peaks at 2-theta (2θ) angle values of 8.99±0.2, 13.52±0.2, 17.54±0.2, 18.07±0.2, 20.74±0.2, 21.79±0.2, 22.57±0.2, and 24.68±0.2.

Even more specifically, the crystalline form of the L-tartrate salt of the present disclosure may further comprise, in an X-ray powder diffraction (XRPD) graph, any one or more diffraction peaks at 2-theta (2θ) angle values selected from the group consisting of 15.64±0.2, 22.88±0.2, 26.32±0.2, and 29.03±0.2.

In addition, the L-tartrate salt of 1-(5-(2-fluorophenyl)-4-methoxy-1-((6-methoxypyridin-3-yl)sulfonyl)-1H-pyrrole-3-yl)-N-methylmethanamine of the present disclosure may exhibit an X-ray powder diffraction spectroscopy pattern of FIG. 18.

The crystalline form of the L-tartrate salt of the present disclosure is characterized by having an endothermic transition peak value at 146 to 158° C. when a temperature increase rate is 20° C./min in a differential scanning calorimetry (DSC) graph, and preferably, characterized by having an endothermic transition peak value at 148 to 156° C., and more preferably at 152±2° C.

In addition, the L-tartrate salt of 1-(5-(2-fluorophenyl)-4-methoxy-1-((6-methoxypyridin-3-yl)sulfonyl)-1H-pyrrole-3-yl)-N-methylmethanamine of the present disclosure may show the differential scanning calorimetry of FIG. 15 or 19.

In addition, the L-tartrate salt of 1-(5-(2-fluorophenyl)-4-methoxy-1-((6-methoxypyridin-3-yl)sulfonyl)-1H-pyrrole-3-yl)-N-methylmethanamine of the present disclosure may have a thermogravimetric analysis (TGA) pattern showing a weight loss of less than 0.1 wt % at 120° C. or less. Specifically, the thermogravimetric analysis (TGA) pattern of FIG. 16 or 20 may be shown.

In the present disclosure, a novel salt that had never been used in the related art was prepared. Specifically, a hydrochloride salt (especially crystalline form I and/or crystalline form II), succinate salt or L-tartrate salt, which is a novel salt of 1-(5-(2-fluorophenyl)-4-methoxy-1-((6-methoxypyridin-3-yl)sulfonyl)-1H-pyrrole-3-yl)-N-methylmethanamine may have excellent stability, photostability, thermal stability, and stability according to pH, thereby being stably maintained without a change in content over a long period of time. In particular, the salt has excellent thermal stability against high temperature, and the like.

Therefore, the raw material of the novel salt of the present disclosure is able to be obtained, and an increase in related substances is remarkably low even when stored for a long time, and thus high purity may be maintained for a long period of time.

In addition, the novel salt of the present disclosure may exhibit excellent pharmacological effects by exhibiting excellent solubility values under various pH conditions, particularly bio-relevant media conditions, and may be usefully used as a new active ingredient of a pharmaceutical composition capable of treating various indications.

According to an embodiment of the present disclosure, when preparing stimulated gastric fluid (SGF), fasted state simulated intestinal fluid (FaSSIF), and fed state simulated intestinal fluid (FeSSIF) and performing a test for measuring solubility and dissolution under conditions close to the in-vivo environment, good solubility was shown. In particular, the solubility in FaSSIF was very good in the hydrochloride salt. From this, it was confirmed that the novel salt according to the present disclosure had significantly excellent solubility in vivo and exhibited high bioavailability.

In addition, since the novel salt exhibits high bioavailability when administered orally, it is possible to exhibit excellent therapeutic effects even when administered in a small amount, thereby significantly improving the patient's medication compliance.

In addition, the novel salt of the present disclosure may have a rapid onset of action and a thermodynamically stable form, and may be very advantageous in processing and storage of pharmaceutical products to achieve easy formulation, and further, may maintain the same state even after the formulation is prepared, so that the uniformity in the formulation content may be stably maintained for a long period of time, and thus the novel salt may be easily applied to mass production.

### Preparation method of novel salt of 1-(5-(2-fluorophenyl)-4-methoxy-1-((6-methoxypyridin-3-yl) sulfonyl)-1H-pyrrole-3-yl)-N-methylmethanamine

There is provided a preparation method of a hydrochloride salt of 1-(5-(2-fluorophenyl)-4-methoxy-1-((6-methoxypyridin-3-yl)sulfonyl)-1H-pyrrole-3-yl)-N-methylmethanamine represented by the following Chemical Formula I.

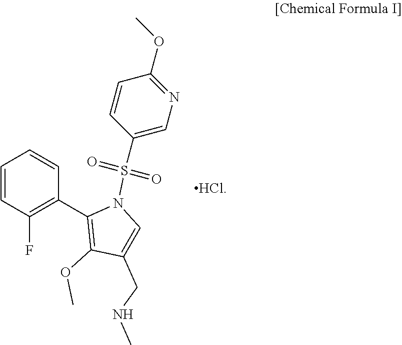 [SMILES: CNCc1cn(S(=O)(=O)c2ccc(OC)nc2)c(-c2ccccc2F)c1OC]

Specifically, the preparation method of the present disclosure comprises:
(1) dissolving a compound represented by the following Chemical Formula IV in a single organic solvent or a mixed solvent to react with hydrochloric acid;

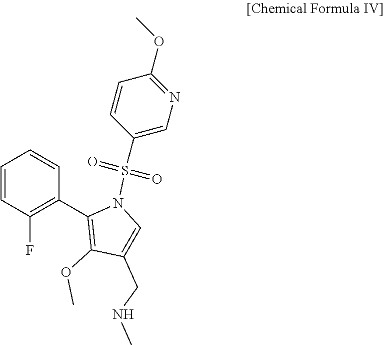 [SMILES: CNCc1cn(S(=O)(=O)c2ccc(OC)nc2)c(-c2ccccc2F)c1OC]

(2) precipitating a product from a reaction solution obtained in step (1); and
(3) filtering and drying the product of step (2).

There is provided a preparation method of a succinate salt of 1-(5-(2-fluorophenyl)-4-methoxy-1-((6-methoxypyridin-3-yl)sulfonyl)-1H-pyrrole-3-yl)-N-methylmethanamine represented by the following Chemical Formula II.

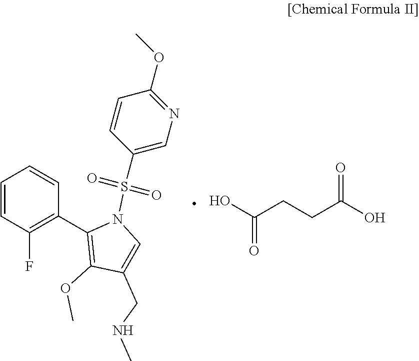 [SMILES: CNCc1cn(S(=O)(=O)c2ccc(OC)nc2)c(-c2ccccc2F)c1OC ; O=C(O)CCC(=O)O]

Specifically, the preparation method of the present disclosure comprises:
(1) dissolving a compound represented by the following Chemical Formula IV in a single organic solvent or a mixed solvent to react with succinic acid;

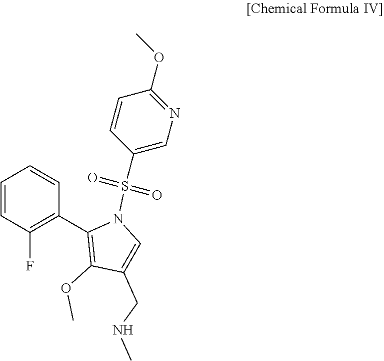 [SMILES: CNCc1cn(S(=O)(=O)c2ccc(OC)nc2)c(-c2ccccc2F)c1OC]

(2) precipitating a product from a reaction solution obtained in step (1); and
(3) filtering and drying the product of step (2).

There is provided a preparation method of an L-tartrate salt of 1-(5-(2-fluorophenyl)-4-methoxy-1-((6-methoxypyridin-3-yl)sulfonyl)-1H-pyrrole-3-yl)-N-methylmethanamine represented by the following Chemical Formula III.

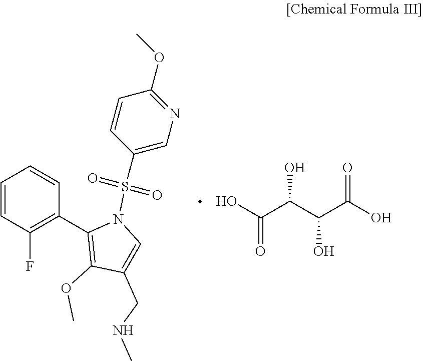 [SMILES: CNCc1cn(S(=O)(=O)c2ccc(OC)nc2)c(-c2ccccc2F)c1OC ; O=C(O)[C@H](O)[C@@H](O)C(=O)O]

Specifically, the preparation method of the present disclosure comprises:
(1) dissolving a compound represented by the following Chemical Formula IV in a single organic solvent or a mixed solvent to react with L-tartaric acid;

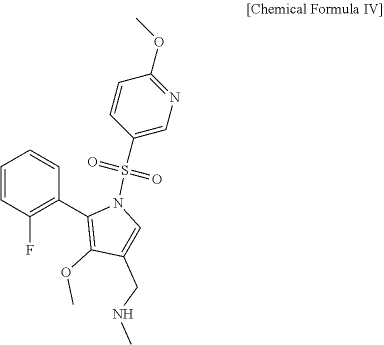 [SMILES: CNCc1cn(S(=O)(=O)c2ccc(OC)nc2)c(-c2ccccc2F)c1OC]

(2) precipitating a product from a reaction solution obtained in step (1); and
(3) filtering and drying the product of step (2).

In the preparation method of Chemical Formula I, II or III of the present disclosure described above, the single organic solvent is preferably one selected from the group consisting of methanol, ethanol, isopropyl alcohol, normal propanol, acetone, methyl ethyl ketone, methyl acetate, ethyl acetate, tetrahydrofuran, 2-methyl tetrahydrofuran and acetonitrile.

In the preparation method of the present disclosure, the mixed solvent is a mixed solvent of (a) at least any one solvent selected from the group consisting of methanol, ethanol, 2-propanol, normal propanol, acetone, methyl ethyl ketone, acetonitrile, methyl acetate, ethyl acetate, tetrahydrofuran, and 2-methyl tetrahydrofuran, and (b) at least any one solvent selected from the group consisting of water, normal heptane, normal hexane, methyl tert-butyl ether (MTBE), THF, diethyl ether, and dimethoxy ethane (DME).

In the preparation of the hydrochloride salt described as Chemical Formula I described above, a mixed solvent of ethanol, isopropanol, acetone or methanol, and MTBE is preferred. When the mixed solvent is used, the crystalline form of the hydrochloride salt has advantages of being stable and being able to be prepared in high yield and high purity.

In the preparation of the succinate salt described as Chemical Formula II described above, a single organic solvent of ethanol, isopropanol, or acetone is preferred. When the single organic solvent is used, the crystalline form of the succinate salt has advantages of being stable and being able to be prepared in high yield and high purity.

In the preparation of the L-tartrate salt described as Chemical Formula III described above, a single organic solvent of ethanol, isopropanol, or acetone is preferred. When the single organic solvent is used, the crystalline form of the L-tartrate salt has advantages of being stable and being able to be prepared in high yield and high purity.

A mixing ratio of the mixed solvent may be 1:1 to 1:20 by volume.

In the preparation method of the present disclosure, preferably, the step (1) may be performed at a temperature of 20 to 40° C., preferably at room temperature.

The hydrochloric acid, succinic acid or L-tartaric acid in step (1) is preferably used in an amount of 0.5 to 2.0 equivalents, and more preferably in an amount of 0.7 to 1.3 equivalents, based on 1.0 equivalent of 1-(5-(2-fluorophenyl)-4-methoxy-1-((6-methoxypyridin-3-yl)sulfonyl)-1H-pyrrole-3-yl)-N-methylmethanamine.

In this step, the reaction may be performed for approximately 12 to 36 hours, preferably 20 to 30 hours, and more preferably 24 hours.

In step (2), the mixture may be cooled to a temperature of 0 to 10° C. or dried under low pressure conditions.

Then, after cooling or drying in step (2), the product may be dried at a temperature of 20 to 70° C. or evaporated under nitrogen stream in step (3). Through the above process, it is possible to effectively remove residual solvents and the like and to obtain the desired crystalline form of the salt in high yield and high purity.

### Pharmaceutical Composition

The present disclosure provides a pharmaceutical composition comprising a hydrochloride salt of 1-(5-(2-fluorophenyl)-4-methoxy-1-((6-methoxypyridin-3-yl)sulfonyl)-1H-pyrrole-3-yl)-N-methylmethanamine represented by the following Chemical Formula I:

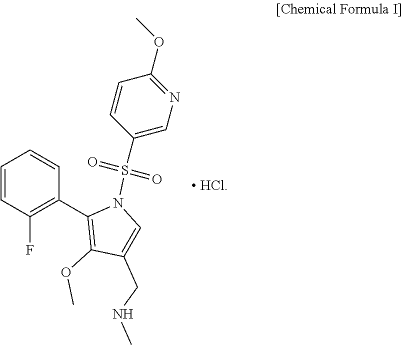

The present disclosure provides a pharmaceutical composition comprising a succinate salt of 1-(5-(2-fluorophenyl)-4-methoxy-1-((6-methoxypyridin-3-yl)sulfonyl)-1H-pyrrole-3-yl)-N-methylmethanamine represented by the following Chemical Formula II:

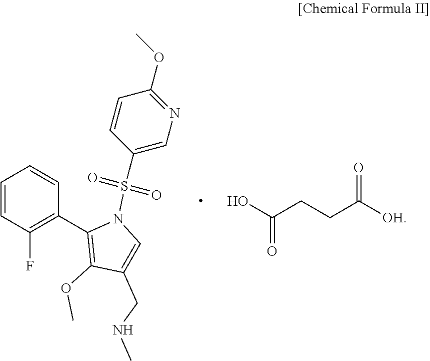 [SMILES: CNCc1cn(S(=O)(=O)c2ccc(OC)nc2)c(-c2ccccc2F)c1OC ; O=C(O)CCC(=O)O]

The present disclosure provides a pharmaceutical composition comprising an L-tartrate salt of 1-(5-(2-fluorophenyl)-4-methoxy-1-((6-methoxypyridin-3-yl)sulfonyl)-1H-pyrrole-3-yl)-N-methylmethanamine represented by the following Chemical Formula III:

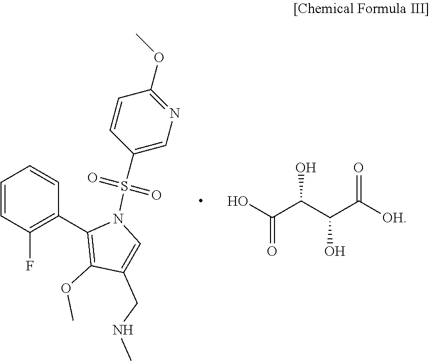 [SMILES: CNCc1cn(S(=O)(=O)c2ccc(OC)nc2)c(-c2ccccc2F)c1OC ; O=C(O)[C@H](O)[C@@H](O)C(=O)O]

The novel salt according to the present disclosure may have excellent stability, photostability, thermal stability, and stability according to pH, and may exhibit excellent solubility in vivo under bio-relevant media conditions close to the in vivo environment, thereby showing excellent pharmacological effects.

Accordingly, the pharmaceutical composition of the present disclosure may be used for the prevention or treatment of gastrointestinal ulcers, gastrointestinal inflammatory diseases or gastric acid-related diseases.

The gastrointestinal ulcer refers to an ulcer occurring in the digestive organs including both the stomach and intestines. Examples of the gastrointestinal ulcer may comprise peptic ulcer, gastric ulcer, duodenal ulcer, NSAID-induced ulcer, acute stress ulcer, Zollinger-Ellison syndrome, and the like, but are not limited thereto. If the ulcer becomes serious, cancer may be developed. For example, in the case of the gastric ulcer, as the degree of the disease becomes severe, the gastric ulcer may develop into gastric cancer.

In particular, the gastrointestinal ulcer may comprise damage to the gastric mucosa or damage to the small intestinal mucosa caused by drugs, alcohol, or the like. In particular, the gastrointestinal ulcer may be damage to the gastric mucosa or damage to the small intestinal mucosa caused by NSAIDs or alcohol.

The gastrointestinal inflammatory disease refers to a disease caused by inflammation of the gastrointestinal tract.

For example, the gastrointestinal inflammatory disease may comprise Helicobacter pylori infection, gastritis (for example, acute hemorrhagic gastritis, chronic superficial gastritis, chronic atrophic gastritis), inflammatory bowel disease, gastric MALT lymphoma, and the like, but is not limited thereto.

The gastric acid-related disease refers to a disease caused by excessive secretion of gastric acid. For example, the gastric acid-related disease may comprise erosive esophagitis, non-erosive esophagitis, reflux esophagitis, symptomatic gastroesophageal reflux disease (symptomatic GERD), functional dyspepsia, hyperacidity, upper gastrointestinal bleeding due to invasive stress, and the like, but is not limited thereto.

According to the present disclosure, the gastrointestinal ulcer, gastrointestinal inflammatory disease or gastric acid-related disease may be any one or more selected from the group consisting of peptic ulcer, gastric ulcer, duodenal ulcer, NSAID-induced ulcer, acute stress ulcer, Zollinger-Ellison syndrome, Helicobacter pylori infection, gastritis, erosive esophagitis, non-erosive esophagitis, reflux esophagitis, inflammatory bowel disease, symptomatic gastroesophageal reflux disease (symptomatic GERD), functional dyspepsia, gastric cancer, gastric MALT lymphoma, hyperacidity, and upper gastrointestinal bleeding due to invasive stress.

The present disclosure provides a pharmaceutical composition comprising a hydrochloride salt of 1-(5-(2-fluorophenyl)-4-methoxy-1-((6-methoxypyridin-3-yl)sulfonyl)-1H-pyrrole-3-yl)-N-methylmethanamine represented by the following Chemical Formula I; and a pharmaceutically acceptable carrier:

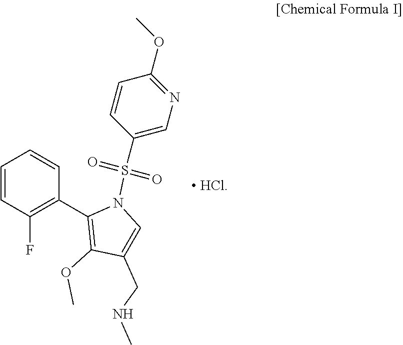

The present disclosure provides a pharmaceutical composition comprising a succinate salt of 1-(5-(2-fluorophenyl)-4-methoxy-1-((6-methoxypyridin-3-yl)sulfonyl)-1H-pyrrole-3-yl)-N-methylmethanamine represented by the following Chemical Formula II; and a pharmaceutically acceptable carrier:

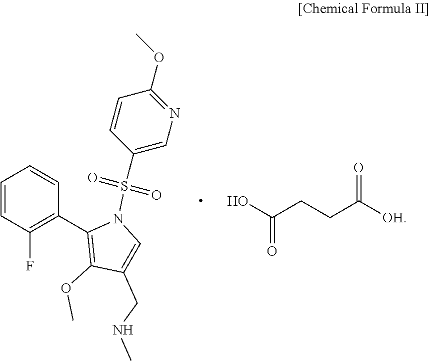 [SMILES: CNCc1cn(S(=O)(=O)c2ccc(OC)nc2)c(-c2ccccc2F)c1OC ; O=C(O)CCC(=O)O]

The present disclosure provides a pharmaceutical composition comprising an L-tartrate salt of 1-(5-(2-fluorophenyl)-4-methoxy-1-((6-methoxypyridin-3-yl)sulfonyl)-1H-pyrrole-3-yl)-N-methylmethanamine represented by the following Chemical Formula III; and a pharmaceutically acceptable carrier:

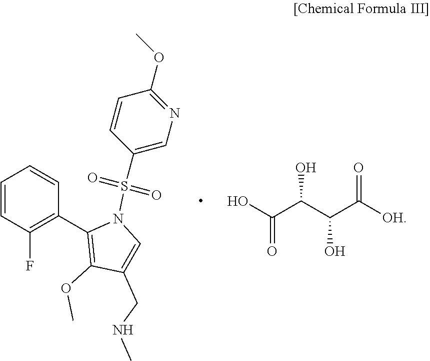 [SMILES: CNCc1cn(S(=O)(=O)c2ccc(OC)nc2)c(-c2ccccc2F)c1OC ; O=C(O)[C@H](O)[C@@H](O)C(=O)O]

In the present disclosure, the “pharmaceutically acceptable carrier” includes any and all solvents, dispersion media, coatings, antibacterial and antifungal agents, isotonic preparations, absorption delaying agents, and the like, that are physiologically compatible.

The composition of the present disclosure may be in various forms. These forms include, for example, liquid, semi-solid, and solid dosage forms, such as liquid solutions (for example, injectable and infusible solutions), dispersions or suspensions, tablets, pills, powders, liposomes and suppositories. The form depends on the intended mode of administration and therapeutic use.

A typical composition is in the form of compositions similar to injectable and infusible solutions. One method of administration is parenteral (for example, intravenous, subcutaneous, intraperitoneal, intramuscular).

The solid dosage forms for oral administration may be provided as, for example, hard or soft capsules, pills, cachets, lozenges or tablets, each containing a predetermined amount of one or more compounds of the present disclosure. In yet another embodiment, the oral administration may be provided in a powder or granular form.

In yet another embodiment, the oral administration may be in a liquid dosage form. The liquid dosage form for oral administration includes, for example, pharmaceutically acceptable emulsions, solutions, suspensions, syrups, and elixirs containing an inert diluent (for example, water) commonly used in the art.

In yet another embodiment, the present disclosure includes a parenteral dosage form. The “parenteral administration” includes, for example, subcutaneous injection, intravenous injection, intraperitoneal injection, intramuscular injection, intrasternal injection, and infusion. The injectable preparation (i.e., sterile injectable aqueous or oleaginous suspension) may be formulated according to known techniques by using suitable dispersants, wetting and/or suspending agents.

Other carrier materials and the administration methods known in the pharmaceutical arts may also be used. The pharmaceutical composition of the present disclosure may be prepared by any of the well-known pharmaceutical techniques, such as effective formulation and administration procedures.

These formulations may be prepared by conventional methods used for formulation in the art or by methods disclosed in Remington's Pharmaceutical Science (latest edition), Mack Publishing Company, and Easton PA, and may be prepared into various formulations according to each disease or an ingredient.

The composition of the present disclosure may be administered orally or parenterally (e.g., intravenously, subcutaneously, intraperitoneally or topically) according to a desired method, and the range of the dose varies depending on the patient's weight, age, sex, health condition, diet, administration time, administration method, excretion rate, severity of the disease, and the like. The daily dose of the novel salt of the present disclosure is about 0.001 to 100 mg/kg, preferably 0.01 to 100 mg/kg, and may be divided and administered once or several times a day.

The pharmaceutical composition of the present disclosure may further comprise at least one active ingredient exhibiting the same or similar medicinal effect in addition to the novel salt.

### Therapeutic Use and Method for Treatment of Gastrointestinal Ulcers, Gastrointestinal Inflammatory Diseases or Gastric Acid-Related Diseases, and Use Thereof in the Manufacture of a Medicament for Treatment

The present disclosure provides a pharmaceutical composition for the prevention or treatment of gastrointestinal ulcers, gastrointestinal inflammatory diseases or gastric acid-related diseases, comprising a hydrochloride salt of 1-(5-(2-fluorophenyl)-4-methoxy-1-((6-methoxypyridin-3-yl)sulfonyl)-1H-pyrrole-3-yl)-N-methylmethanamine represented by the following Chemical Formula I:

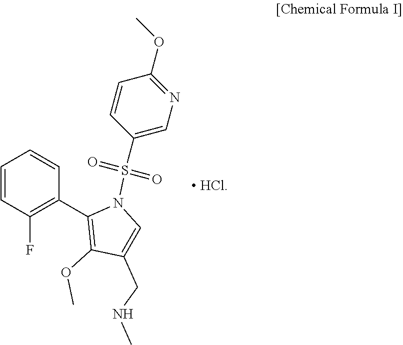

The present disclosure provides a method for treating gastrointestinal ulcers, gastrointestinal inflammatory diseases or gastric acid-related diseases, comprising administering to a subject in need thereof a therapeutically effective amount of the hydrochloride salt of 1-(5-(2-fluorophenyl)-4-methoxy-1-((6-methoxypyridin-3-yl)sulfonyl)-1H-pyrrole-3-yl)-N-methylmethanamine.

The present disclosure provides a pharmaceutical composition for the prevention or treatment of gastrointestinal ulcers, gastrointestinal inflammatory diseases or gastric acid-related diseases, comprising a succinate salt of 1-(5-(2-fluorophenyl)-4-methoxy-1-((6-methoxypyridin-3-yl)sulfonyl)-1H-pyrrole-3-yl)-N-methylmethanamine represented by the following Chemical Formula II:

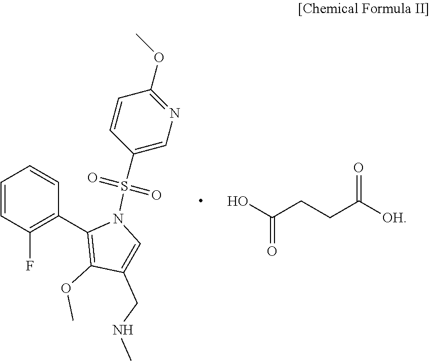 [SMILES: CNCc1cn(S(=O)(=O)c2ccc(OC)nc2)c(-c2ccccc2F)c1OC ; O=C(O)CCC(=O)O]

The present disclosure provides a method for treating gastrointestinal ulcers, gastrointestinal inflammatory diseases or gastric acid-related diseases, comprising administering to a subject in need thereof a therapeutically effective amount of the succinate salt of 1-(5-(2-fluorophenyl)-4-methoxy-1-((6-methoxypyridin-3-yl)sulfonyl)-1H-pyrrole-3-yl)-N-methylmethanamine.

The present disclosure provides a pharmaceutical composition for the prevention or treatment of gastrointestinal ulcers, gastrointestinal inflammatory diseases or gastric acid-related diseases, comprising an L-tartrate salt of 1-(5-(2-fluorophenyl)-4-methoxy-1-((6-methoxypyridin-3-yl)sulfonyl)-1H-pyrrole-3-yl)-N-methylmethanamine represented by the following Chemical Formula III:

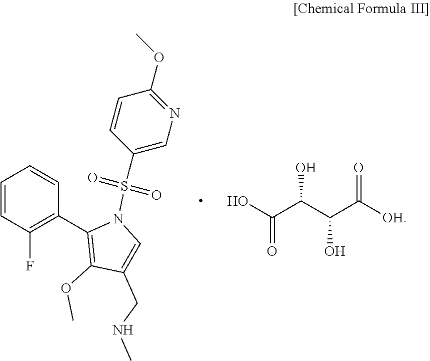 [SMILES: CNCc1cn(S(=O)(=O)c2ccc(OC)nc2)c(-c2ccccc2F)c1OC ; O=C(O)[C@H](O)[C@@H](O)C(=O)O]

The present disclosure provides a method for treating gastrointestinal ulcers, gastrointestinal inflammatory diseases or gastric acid-related diseases, comprising administering to a subject in need thereof a therapeutically effective amount of the L-tartrate salt of 1-(5-(2-fluorophenyl)-4-methoxy-1-((6-methoxypyridin-3-yl)sulfonyl)-1H-pyrrole-3-yl)-N-methylmethanamine.

The subject in need thereof means mammals including a human, which includes mammals such as humans, monkeys, cattle, horses, dogs, cats, rabbits, rats, and mice.

As used herein, the term “therapeutically effective amount” refers to an amount of the novel salt effective for the prevention or treatment of gastrointestinal ulcers, gastrointestinal inflammatory diseases, or gastric acid-related diseases, or a pharmaceutical composition comprising the same. For example, the therapeutically effective amount may include, as an amount of the novel salt to be administered to the subject to be treated, any amount of the pharmaceutical composition including the above-described salts, to prevent occurrence or recurrence of gastrointestinal ulcers, gastrointestinal inflammatory diseases, or gastric acid-related diseases, alleviate symptoms, inhibit direct or indirect pathological results, prevent metastasis, reduce the rate of progression, alleviate or temporarily ameliorate the condition or improve the prognosis. In other words, the therapeutically effective amount may be interpreted to encompass all doses in which symptoms of gastrointestinal ulcers, gastrointestinal inflammatory diseases, or gastric acid-related disease are improved or cured by the pharmaceutical composition.

The method for preventing or treating gastrointestinal ulcers, gastrointestinal inflammatory diseases, or gastric acid-related diseases of the present disclosure includes not only treating the diseases themselves before the onset of signs, but also inhibiting or avoiding signs thereof by administering the above-described salt or the pharmaceutical composition comprising the same. In the management of the diseases, a prophylactic or therapeutic dose of a specific active ingredient will vary according to the nature and severity of the disease or condition, and a route by which the active ingredient is administered. The dose and frequency of dose will vary depending on the age, weight and response of individual patients. A suitable dosage regimen may be readily selected by one of ordinary skill in the art considering these factors naturally. In addition, the method for treating gastrointestinal ulcers, gastrointestinal inflammatory diseases or gastric acid-related diseases using the pharmaceutical composition of the present disclosure may further comprise administering a therapeutically effective amount of an additional active agent useful for treating the diseases together with the above-described salt, and the additional active agent may exhibit synergistic or auxiliary effects with the above-described salt which is an active ingredient according to the present disclosure.

The present disclosure also provides use of a hydrochloride salt, succinate salt or L-tartrate salt of 1-(5-(2-fluorophenyl)-4-methoxy-1-((6-methoxypyridin-3-yl)sulfonyl)-1H-pyrrole-3-yl)-N-methylmethanamine for the preparation of a drug for treatment of gastrointestinal ulcers, gastrointestinal inflammatory diseases or gastric acid-related diseases.

The above-described salts for the preparation of the drug may be mixed with acceptable adjuvants, diluents, carriers, and the like, and may be prepared as a combined preparation together with other active agents to have a synergistic action of the active ingredients.

The matters mentioned in the uses, compositions and treatment methods of the present disclosure are applied equally unless they contradict each other.

### Advantageous Effects

The novel salt according to the present disclosure may have excellent stability to increase the stability of the formulation and may have improved solubility (particularly, solubility in vivo) and bioavailability and thus can be usefully used as an active ingredient of a pharmaceutical composition.

### DESCRIPTION OF DRAWINGS

The following drawings attached to the present specification illustrate preferred embodiments of the present disclosure, and serve to further understand the technical idea of the present disclosure together with the above-described content of the invention, and thus the present disclosure should not be construed as being limited only to the matters described in these drawings.

FIG. 1 is an X-ray powder diffraction analysis (XRPD) graph of a crystalline form I of a hydrochloride salt compound represented by Chemical Formula I prepared in Example 1 of the present disclosure.

FIG. 2 is a differential scanning calorimetry (DSC) pattern analysis graph of the crystalline form I of the hydrochloride salt compound represented by Chemical Formula I prepared in Example 1 of the present disclosure.

FIG. 3 is a thermogravimetric analysis (TGA) pattern analysis graph of the crystalline form I of the hydrochloride salt compound represented by Chemical Formula I prepared in Example 1 of the present disclosure.

FIG. 4 shows ¹H-NMR results for confirming ratios of cations and anions of the crystalline form I of the hydrochloride salt compound represented by Chemical Formula I prepared in Example 1 of the present disclosure.

FIG. 5 is an X-ray powder diffraction analysis (XRPD) graph of a crystalline form II of a hydrochloride salt compound represented by Chemical Formula I prepared in Example 2 of the present disclosure.

FIG. 6 is a differential scanning calorimetry (DSC) pattern analysis graph of the crystalline form II of the hydrochloride salt compound represented by Chemical Formula I prepared in Example 2 of the present disclosure.

FIG. 7 is a thermogravimetric analysis (TGA) pattern analysis graph of the crystalline form II of the hydrochloride salt compound represented by Chemical Formula I prepared in Example 2 of the present disclosure.

FIG. 8 is a differential scanning calorimetry (DSC) pattern analysis graph of a succinate salt compound represented by Chemical Formula II prepared in Example 4 of the present disclosure.

FIG. 9 is a thermogravimetric analysis (TGA) pattern analysis graph of the succinate salt compound represented by Chemical Formula II prepared in Example 4 of the present disclosure.

FIG. 10 shows ¹H-NMR results for confirming ratios of cations and anions of the succinate salt compound represented by Chemical Formula II prepared in Example 4 of the present disclosure.

FIG. 11 is an X-ray powder diffraction analysis (XRPD) graph of a succinate salt compound represented by Chemical Formula II prepared in Example 6 of the present disclosure.

FIG. 12 is a differential scanning calorimetry (DSC) pattern analysis graph of the succinate salt compound represented by Chemical Formula II prepared in Example 6 of the present disclosure.

FIG. 13 is a thermogravimetric analysis (TGA) pattern analysis graph of the succinate salt compound represented by Chemical Formula II prepared in Example 6 of the present disclosure.

FIG. 14 shows ¹H-NMR results for confirming ratios of cations and anions of the succinate salt compound represented by Chemical Formula II prepared in Example 6 of the present disclosure.

FIG. 15 is a differential scanning calorimetry (DSC) pattern analysis graph of an L-tartrate salt compound represented by Chemical Formula III prepared in Example 7 of the present disclosure.

FIG. 16 is a thermogravimetric analysis (TGA) pattern analysis graph of the L-tartrate salt compound represented by Chemical Formula III prepared in Example 7 of the present disclosure.

FIG. 17 shows ¹H-NMR results for confirming ratios of cations and anions of the L-tartrate salt compound represented by Chemical Formula III prepared in Example 7 of the present disclosure.

FIG. 18 is an X-ray powder diffraction analysis (XRPD) graph of an L-tartrate salt compound represented by Chemical Formula III prepared in Example 8 of the present disclosure.

FIG. 19 is a differential scanning calorimetry (DSC) pattern analysis graph of the L-tartrate salt compound represented by Chemical Formula III prepared in Example 8 of the present disclosure.

FIG. 20 is a thermogravimetric analysis (TGA) pattern analysis graph of the L-tartrate salt compound represented by Chemical Formula III prepared in Example 8 of the present disclosure.

FIG. 21 shows ¹H-NMR results for confirming ratios of cations and anions of the L-tartrate salt compound represented by Chemical Formula III prepared in Example 8 of the present disclosure.

FIGS. 22a and 22b are a graph showing results of dynamic vapor sorption (DVS) measurement of the hydrochloride salt compound represented by Chemical Formula I prepared in Example 1 of the present disclosure.

FIGS. 23a and 23b are a graph showing results of dynamic vapor sorption (DVS) measurement of the L-tartrate salt compound represented by Chemical Formula III prepared in Example 8 of the present disclosure.

### BEST MODE

Hereinafter, Examples and the like will be described in detail to assist the understanding of the present disclosure. However, these Examples according to the present disclosure may be modified in various other forms, and the scope of the present disclosure should not be construed as being limited to the following Examples. These Examples of the present disclosure are provided to more fully explain the present disclosure to those of ordinary skill in the art.

Instrument used, samples and measurement conditions

### 1) XRPD (X-ray Powder Diffractometer)

The X-ray powder diffraction (XRPD) pattern was measured using an XRD-6000 instrument manufactured by Shimadzu Corporation, and the conditions of use were set as shown in Table 1 below.

TABLE 1

| Setting | Parameters |
|---|---|
| Tube: | Cu: K-Alpha (λ = 1.54056 Å) |
| Generator: | Voltage: 40 KV Current: 30 mA |
| Scan Scope: | 2-50° (degree) |
| Scan Scope: | 5°/min |

### 2) DSC (Differential Scanning Calorimeter)

Using DSC3 manufactured by Mettler Toledo Corporation, a compound sample (about 1 mg) was tested in a pinhole aluminum pan under nitrogen purge at a ramp rate of 20° C./min over the range of 30 to 300° C.

Specific conditions were set as shown in Table 2 below.

TABLE 2

| Setting | Parameters |
|---|---|
| Ramp rate | 20° C./min, over the range 30° C.~300° C. |
| Nitrogen purge | 50 mL/min |
| Samples weight | ~1 mg |

### 3) TGA (Thermal Gravimetric Analysis)

Using Pyris 1 TGA manufactured by PerkinElmer, Inc., a compound sample (about 5 mg) was weighed in pans under nitrogen purge at a ramp rate of 20° C./min over the range of 30 to 300° C.

Specific conditions were set as shown in Table 3 below.

TABLE 3

| Setting | Parameters |
|---|---|
| Ramp rate | 20° C./min, over the range 30° C.~300° C. |
| Nitrogen purge | 50 mL/min |
| Samples weight | ~5 mg |

### 4) 1H-NMR (Nuclear Magnetic Resonance)

About 3 mg of the compound was weighed into a nuclear magnetic tube and 0.5 mL of deuterated dimethyl sulfoxide was added to completely dissolve the sample. The tube was put in a rotor and placed in the open position of an auto sampler and scanned with a BRUKER AVANCE III (400 MHz).

### 5) HPLC (High-Performance Liquid Chromatography)

HPLC conditions for measuring the solubility of compounds were shown in Table 4 below.

TABLE 4

| Column | C18, 150 × 4.6 mm, 5 μm | |
|---|---|---|
| Mobile phase A: | 0.05% TFA in H₂O | |
| Mobile phase B: | 0.05% TFA in ACN | |
| Gradient: | 0.00 min | 5% B |
| | 6.50 min | 90% B |
| | 7.00 min | 5% B |
| | 10.00 min | 5% B |

| | |
|---|---|
| Column temperature: | 40° C. |
| Flow rate: | 1.0 ml/minute |
| Detector wavelength: | 220 nm |
| Injection volume: | 5 μL |
| Diluent: | MeOH |

HPLC conditions for measuring the stability of compounds were shown in Table 5 below.

TABLE 5

| | |
|---|---|
| Column | C18, 150 × 4.6 mm, 5 μm |
| Mobile phase A: | 0.05% TFA in H₂O |
| Mobile phase B: | 0.05% TFA in ACN |
| Gradient: | 0.00 min |

| | |
|---|---|
| 20.00 min | 90% B |
| 25.00 min | 90% B |
| 25.10 min | 5% B |
| 30.00 min | 5% B |

| | |
|---|---|
| Column | 40° C. |
| temperature: | |
| Flow rate: | 1.0 ml/minute |
| Detector | 220 nm |
| wavelength: | |
| Injection volume: | 5 μL |
| Diluent: | MeOH |

### 6) DVS (Dynamic Vapor Sorption)

Using DVS intrinsic manufactured by SMS Co., Ltd, about 20 mg of the sample was tested for water sorption/desorption profiles at 25° C. under a 0%˜95%˜0% relative humidity (RH) cycle using the following parameters.
Temperature: T=25° C.
Equilibrium: dm/dt: 0.01%/min.
Range of RH (%) measurement step: 0%˜95%˜0%; RH(%) measurement step: 5%

### 7) Preparation of 1-(5-(2-fluorophenyl)-4-methoxy-1-((6-methoxypyridin-3-yl)sulfonyl)-1H-pyrrole-3-yl)-N-methylmethanamine Free Base

### Step (1): Synthesis of methyl 5-(2-fluorophenyl)-4-methoxy-1-((6-methoxypyridin-3-yl)sulfonyl)-1H-pyrrole-3-carboxylate

Methyl 5-(2-fluorophenyl)-4-methoxy-1H-pyrrole-3-carboxylate (intermediate 1, 1.0 eq., 920 g, 3.69 mol) was dissolved in DMF (9.2 L), and then added dropwise with t-BuOK (2.0 eq., 828 g, 7.38 mmol) at 0° C. and stirred for 30 minutes. 6-methoxypyridine-3-sulfonyl chloride (1.5 eq., 1.15 kg, 5.54 mol) was added, followed by stirring at 0° C. for 1 hour. The reaction solution was added with water, and then extracted with ethyl acetate. The organic layer was dried over anhydrous magnesium sulfate, filtered, concentrated, and purified by column chromatography to obtain methyl 5-(2-fluorophenyl)-4-methoxy-1-((6-methoxypyridin-3-yl)sulfonyl)-1H-pyrrole-3-carboxylate as a white solid (1.20 kg, 77.4%).

### Step (2): Synthesis of 5-(2-fluorophenyl)-4-methoxy-1-((6-methoxypyridin-3-yl)sulfonyl)-1H-pyrrole-3-yl)methanol

Methyl 5-(2-fluorophenyl)-4-methoxy-1-((6-methoxypyridin-3-yl)sulfonyl)-1H-pyrrole-3-carboxylate (1.0 eq., 1.1 kg, 2.62 mol) was dissolved in THF (11.0 mL), and DIBAL 2.0 M in THF solution (3.0 eq., 3.93 L, 7.86 mol) was added dropwise at 0° C., followed by stirring for 30 minutes. The reaction was completed with a 5% aqueous Rochelle salt solution in the reaction solution and the reaction solution was extracted with ethyl acetate. The organic layer was dried over anhydrous magnesium sulfate, filtered, and concentrated to obtain 5-(2-fluorophenyl)-4-methoxy-1-((6-methoxypyridin-3-yl)sulfonyl)-1H-pyrrole-3-yl)methanol as a light yellow oil (870 g, 84.8%).

### Step (3): Synthesis of 5-(2-fluorophenyl)-4-methoxy-1-((6-methoxypyridin-3-yl) sulfonyl)-1H-pyrrole-3-carbaldehyde

5-(2-fluorophenyl)-4-methoxy-1-((6-methoxypyridin-3-yl)sulfonyl)-1H-pyrrole-3-yl)methanol (1.0 eq., 830 g, 2.12 mol) and TEA (4.0 eq., 1.59 kg, 15.7 mol) were dissolved in dimethyl sulfoxide (DMSO) (4.15 L), and then added dropwise with SO₃-pyridine (4.0 eq., 1.35 kg, 8.48 mol) and stirred at room temperature for 1.5 hours. The reaction product was added with water at 0° C., and then extracted with ethyl acetate. The organic layer was dried over anhydrous magnesium sulfate, filtered, and concentrated to obtain (5-(2-fluorophenyl)-4-methoxy-1-((6-methoxypyridin-3-yl)sulfonyl)-1H-pyrrole-3-carbaldehyde as a yellow solid (722 g, 87.6%).

### Step (4): Synthesis of 1-(5-(2-fluorophenyl)-4-methoxy-1-((6-methoxypyridin-3-yl) sulfonyl)-1H-pyrrole-3-yl)-N-methylmethanamine

5-(2-fluorophenyl)-4-methoxy-1-((6-methoxypyridin-3-yl)sulfonyl)-1H-pyrrole-3-carbaldehyde (1.0 eq., 715 g, 1.83 mol) was dissolved in methanol (7.2 L), and added with methylamine in methanol (5.0 eq., 916 g, 9.16 mol). After stirring at room temperature for 1 hour, the reaction product was concentrated, dissolved in ethanol (7.2 L), cooled to 0° C., and then added with NaBH₄ (2.0 eq., 139 g, 3.66 mol) and stirred at 0° C. for 1 hour. The reaction solution was added with water and extracted with ethyl acetate. The organic layer was dried over anhydrous magnesium sulfate, filtered, concentrated, and purified by column chromatography to obtain 1-(5-(2-fluorophenyl)-4-methoxy-1-((6-methoxypyridin-3-yl) sulfonyl)-1H-pyrrole-3-yl)-N-methylmethanamine as a brown oil (347 g, 46.7%).

TABLE 6

| | | HPLC | |
|---|---|---|---|
| | | Reten- | LC-MS |
| | | tion | value |
| | | Time | [M + |
| Compound name | NMR chemical shift | (min) | H]⁺ |
| 1-(5-(2- | ¹H NMR (400 MHz, CDCl₃) δ | 9.067 | 405 |
| fluorophenyl)-4- | 8.12 (d, J = 2.4 Hz, 1H), | | |
| methoxy-1-((6- | 7.67-7.65 (m, 2H), 7.47- | | |
| methoxypyridin-3- | 7.43 (m, 1H), 7.25 (dt, | | |
| yl)sulfonyl)-1H- | J = 7.3, 1.7 Hz, 1H), 7.18 | | |
| pyrrole-3-yl)-N- | (t, J = 7.4 Hz, 1H), 7.05 | | |
| methylmethanamine | (t, J = 8.8 Hz, 1H), 6.73 | | |
| | (d, J = 8.8 Hz, 1H), 3.99 | | |
| | (s, 3H), 3.94 (s, 2H), 3.46 | | |
| | (s, 3H), 2.64 (s, 3H). | | |

### <Example 1> Preparation of hydrochloride salt of 1-(5-(2-fluorophenyl)-4-methoxy-1-((6-methoxypyridin-3-yl) sulfonyl)-1H-pyrrole-3-yl)-N-methylmethanamine

About 500 mg of 1-(5-(2-fluorophenyl)-4-methoxy-1-((6-methoxypyridin-3-yl)sulfonyl)-1H-pyrrole-3-yl)-N-methylmethanamine free base was weighed and put into a glass vial, and then dissolved in 2 mL of ethanol while heating at 25° C. Then, 647.44 μL (2 M) of hydrochloric acid was added to the vial. The sample was stirred continuously for 24 hours in a magnetic stirrer under room temperature conditions, and after stirring for 24 hours, the solid precipitate was separated by centrifugation. Then, the wet solid was dried at 40° C. for 20 hours, and then a light gray dried powder was obtained.

The resulting salt was analyzed using XRPD, DSC, TGA and ¹H-NMR.

A brief summary of the results of the analysis above was shown in Table 7 below.

TABLE 7

| | | TGA weight | ¹H-NMR (Ratio of |
|---|---|---|---|
| XRPD | DSC | loss (% w/w) | cation and anion) |
| Crystalline | Melting onset: | Weight | 1:1 |
| Form I | 181.30° C., | loss: ~0.17%, | |
| | Endset: 186.94° C. | 120° C. | |
| | Peak: 183.66° C. | | |
| | (79.01 J/g) | | |

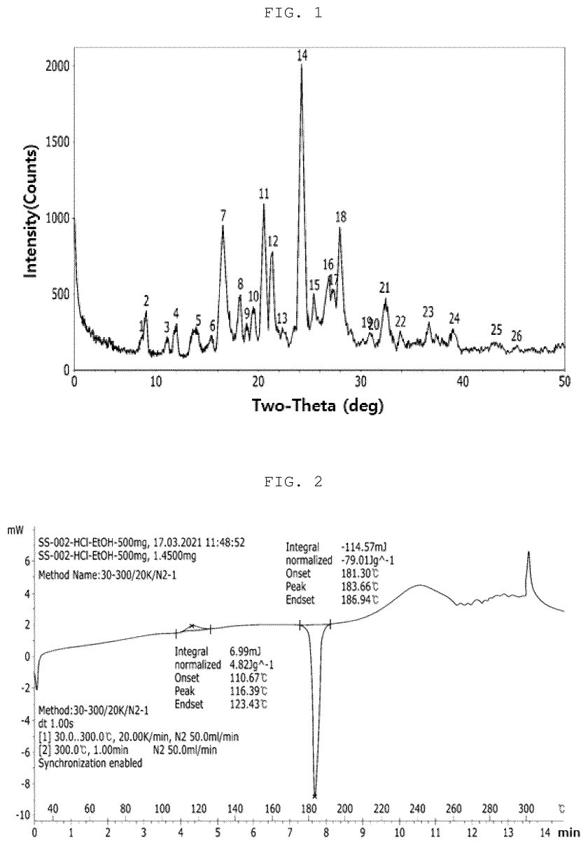

The XRPD analysis results were shown in FIG. 1.

As a result of measuring XRPD of the prepared compounds, the values of FIG. 1 were confirmed. Specifically, XRPD patterns with peaks were shown at 8.97, 16.53, 18.15, 20.53, 21.32, 24.25, 25.45, 26.78, 27.32, 28.04, and 32.40° 2θ±0.2°. In particular, characteristic peaks were identified at 16.53, 20.53, 21.32, 24.25, 26.78, 27.32, and 28.040 2θ±0.20.

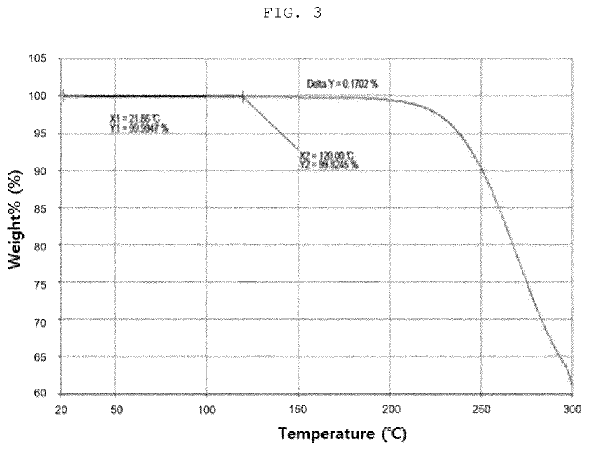

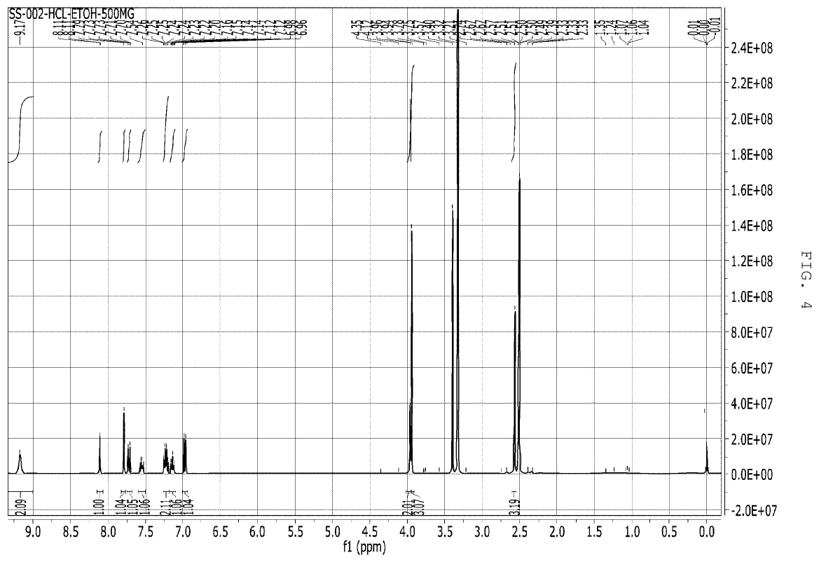

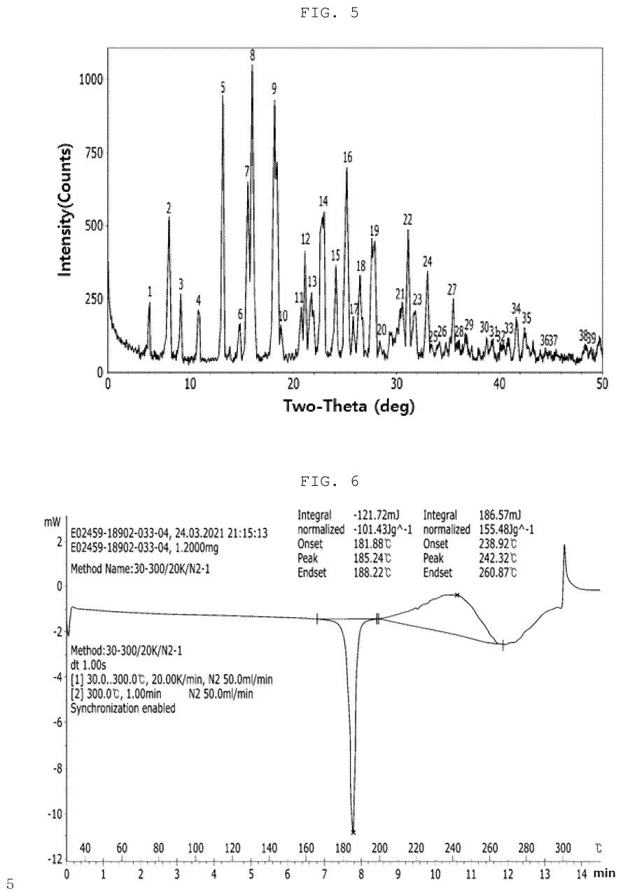

The results of DSC, TGA, and ¹H-NMR analysis were shown in FIGS. 2, 3 and 4, respectively.

As can be confirmed through the experiment, it was confirmed that the melting point was 183.66° C. and the TGA was about 0.17% to secure increased stability of materials and ease of storage. In other words, through the results, the production of the crystalline form was confirmed and it was confirmed that not only the content of residual solvent was low, but also a melting point (MP) value suitable for commercial use was measured.

### <Example 2> Preparation of hydrochloride salt of 1-(5-(2-fluorophenyl)-4-methoxy-1-((6-methoxypyridin-3-yl) sulfonyl)-1H-pyrrole-3-yl)-N-methylmethanamine

430 g of 1-(5-(2-fluorophenyl)-4-methoxy-1-((6-methoxypyridin-3-yl)sulfonyl)-1H-pyrrole-3-yl)-N-methylmethanamine was dissolved in methanol (430 mL), and added with hydrochloric acid in methanol (2 N, 1.0 L). Additionally, MTBE (4.3 L) was slowly added dropwise at room temperature for 0.5 hours. After stirring at room temperature for 1 hour, the resulting solid was filtered. Then, the wet solid was dried at 45° C. for 6 hours, and then a light gray dried powder was obtained (415 g, 88.5%).

A brief summary of the results of the analysis above was shown in Table 8 below.

TABLE 8

| | | TGA weight | ¹H-NMR (Ratio of |
|---|---|---|---|
| XRPD | DSC | loss (% w/w) | cation and anion) |
| Crystalline | Melting onset: | Weight | 1:1 |
| Form II | 181.88° C. | loss: ~0.04% | |
| | Endset: 188.22° C. | 120° C. | |
| | Peak: 185.24° C. | | |
| | (101.43 J/g) | | |

The XRPD analysis results were shown in FIG. 5.

As a result of measuring XRPD of the prepared compounds, the values of FIG. 5 were confirmed. Specifically, XRPD patterns with peaks were shown at 7.96, 13.22, 15.64, 16.04, 18.16, 21.12, 22.88, 24.12, 25.18, 27.80, and 31.12° 2θ±0.2°. In particular, characteristic peaks were identified at 7.96, 13.22, 15.64, 16.04, 18.16, 22.88, and 25.180 2θ±0.20.

The DSC and TGA analysis results were shown in FIGS. 6 and 7, respectively.

As can be confirmed through the experiment, it was confirmed that the melting point was 185.24° C. and the TGA was about 0.04% to secure increased stability of materials and ease of storage. In other words, through the results, the production of the crystalline form was confirmed and it was confirmed that not only the content of residual solvent was low, but also a melting point (MP) value suitable for commercial use was measured.

### <Example 3> Preparation of hydrochloride salt of 1-(5-(2-fluorophenyl)-4-methoxy-1-((6-methoxypyridin-3-yl) sulfonyl)-1H-pyrrole-3-yl)-N-methylmethanamine

About 50 mg of 1-(5-(2-fluorophenyl)-4-methoxy-1-((6-methoxypyridin-3-yl)sulfonyl)-1H-pyrrole-3-yl)-N-methylmethanamine free base was weighed and put into a glass vial, and then dissolved in 0.6 mL of isopropyl alcohol at 25° C. Then, 64.74 μL of hydrochloric acid (2 M in MeOH) was added to the vial. The sample was stirred continuously for 24 hours in a magnetic stirrer under room temperature conditions, and after stirring for 24 hours, a solid precipitate was separated by centrifugation. Next, the wet solid was further dried under low pressure conditions at room temperature, and the dried solid was analyzed.

The resulting salt was analyzed using XRPD, and the same results as the crystalline form prepared under the ethanol solvent were obtained.

### <Example 4> Preparation of succinate salt of 1-(5-(2-fluorophenyl)-4-methoxy-1-((6-methoxypyridin-3-yl) sulfonyl)-1H-pyrrole-3-yl)-N-methylmethanamine

About 50 mg of 1-(5-(2-fluorophenyl)-4-methoxy-1-((6-methoxypyridin-3-yl)sulfonyl)-1H-pyrrole-3-yl)-N-methylmethanamine free base was weighed and put into a glass vial, and then dissolved in 0.6 mL of isopropyl alcohol at 25° C. Then, 129.49 μL of succinic acid (2 M in MeOH) was added to the vial. The sample was stirred continuously for 24 hours in a magnetic stirrer under room temperature conditions, and after stirring for 24 hours, a solid precipitate was separated by centrifugation. Next, the wet solid was further dried under low pressure conditions at room temperature, and the dried solid was analyzed.

The resulting salt was analyzed using XRPD, DSC, TGA and ¹H-NMR.

A brief summary of the results of the analysis above was shown in Table 9 below.

TABLE 9

| | | TGA weight | ¹H-NMR (Ratio of |
|---|---|---|---|
| XRPD | DSC | loss (% w/w) | cation and anion) |
| Crystalline | Melting onset: | Weight | 1:1 |
| Form | 138.57° C. | loss: ~0.05% | |
| | Endset: 143.58° C. | 120° C. | |
| | Peak: 140.83° C. | | |
| | (88.94 J/g) | | |

As a result of XRPD analysis, it was confirmed that one crystalline form was produced.

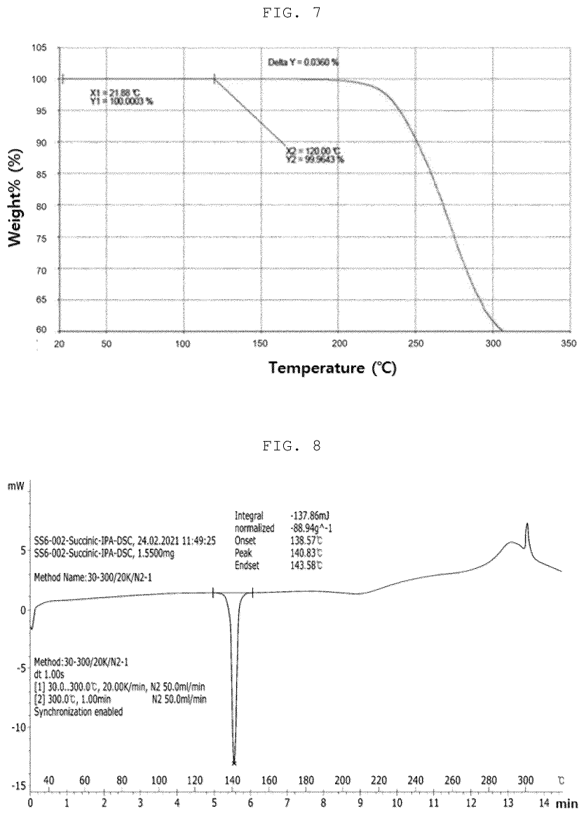

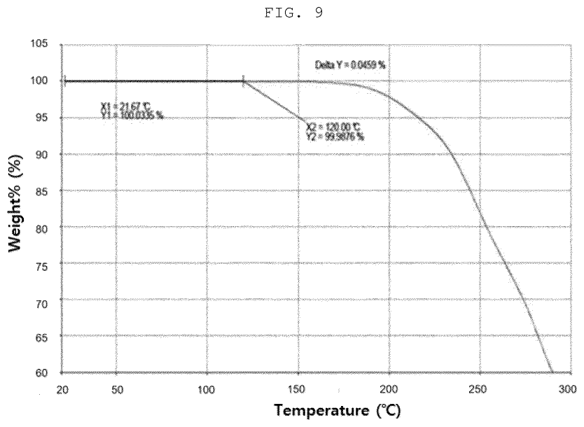

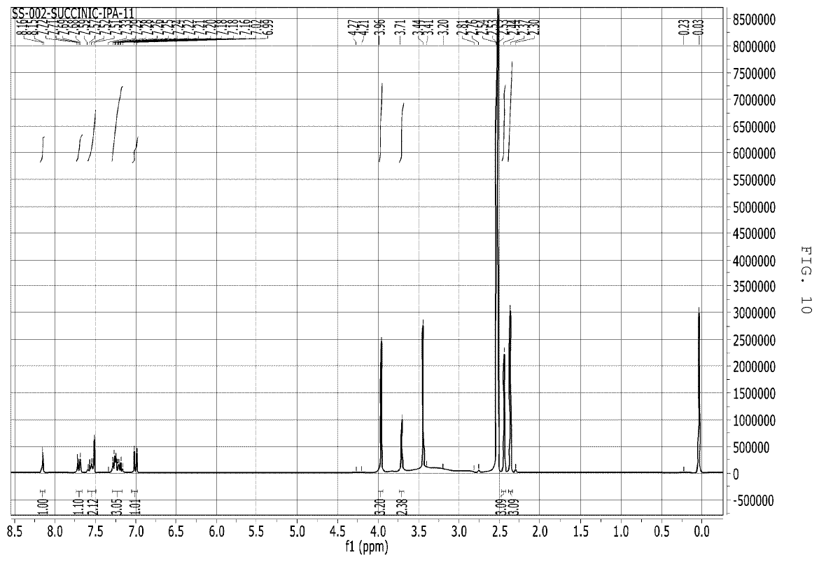

The results of DSC, TGA, and ¹H-NMR analysis were shown in FIGS. 8, 9 and 10, respectively.

As can be confirmed through the experiment, it was confirmed that the melting point was 140.83° C. and the TGA was about 0.05% to secure increased stability of materials and ease of storage.

### <Example 5> Preparation of succinate salt of 1-(5-(2-fluorophenyl)-4-methoxy-1-((6-methoxypyridin-3-yl) sulfonyl)-1H-pyrrole-3-yl)-N-methylmethanamine

80.13 mg of 1-(5-(2-fluorophenyl)-4-methoxy-1-((6-methoxypyridin-3-yl)sulfonyl)-1H-pyrrole-3-yl)-N-methylmethanamine free base was weighed and put into a glass vial, and then dissolved in 0.4 mL of acetone while heating at 25° C. Then, 207.18 μL of succinic acid (1 M in MeOH) was added to the vial. The sample was stirred continuously for 24 hours in a magnetic stirrer under room temperature conditions, and after stirring for 24 hours, a solid precipitate was separated by centrifugation and dried at 40° C. for 12 hours.

The resulting salt was analyzed using XRPD, and the same results as the crystalline form prepared under the isopropyl alcohol solvent were obtained.

### <Example 6> Preparation of succinate salt of 1-(5-(2-fluorophenyl)-4-methoxy-1-((6-methoxypyridin-3-yl) sulfonyl)-1H-pyrrole-3-yl)-N-methylmethanamine

About 500 mg of 1-(5-(2-fluorophenyl)-4-methoxy-1-((6-methoxypyridin-3-yl)sulfonyl)-1H-pyrrole-3-yl)-N-methylmethanamine free base was weighed and put into a glass vial, and then dissolved in 2 mL of ethanol while heating at 25° C. Then, 1356.55 μL (1 M) of succinic acid was added to the vial. The sample was stirred continuously for 24 hours in a magnetic stirrer under room temperature conditions, and after stirring for 24 hours, a solid precipitate was separated by centrifugation. Then, the wet solid was dried at 40° C. for 20 hours, and then a light gray dried powder was obtained.

The resulting salt was analyzed using XRPD, DSC, TGA and ¹H-NMR.

A brief summary of the results of the analysis above was shown in Table 10 below.

TABLE 10

| | | TGA weight | ¹H-NMR (Ratio of |
|---|---|---|---|
| XRPD | DSC | loss (% w/w) | cation and anion) |
| Crystalline | Melting onset: | Weight | 1:1 |
| Form | 139.18° C. | loss: ~0.10%, | |
| | Endset: 144.36° C. | 120° C. | |
| | Peak: 141.04° C. | | |
| | (99.62 J/g) | | |

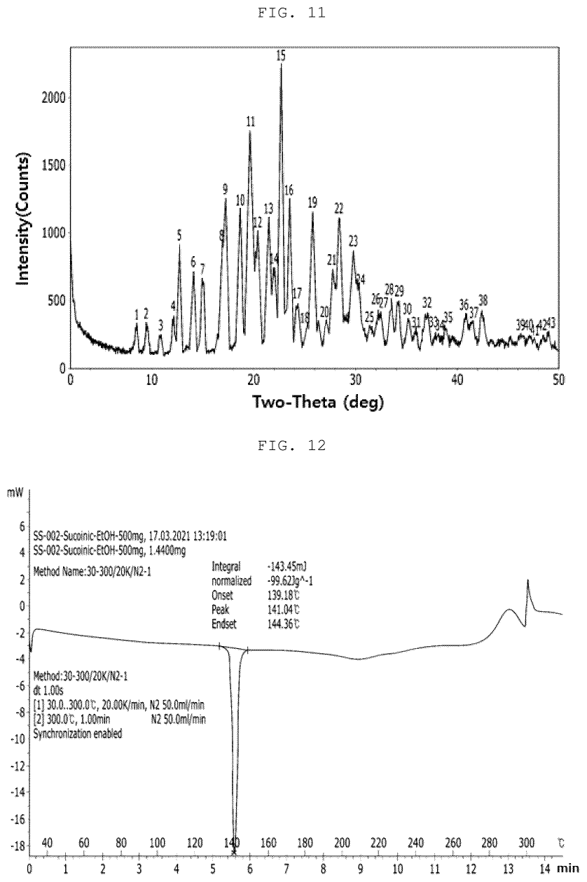

The XRPD analysis results were shown in FIG. 11.

As a result of measuring XRPD of the prepared compounds, the values of FIG. 11 were confirmed.

Specifically, XRPD patterns with peaks were shown at 12.75, 14.09, 15.02, 17.14, 18.70, 19.74, 20.42, 21.51, 22.75, 23.53, 25.81, 28.40, and 29.82° 2θ±0.2°. In particular, characteristic peaks were identified at 17.14, 18.70, 19.74, 21.51, 22.75, 23.53, 25.81, and 28.400 2θ±0.2°.

The confirmed crystal form showed the same XRPD pattern value as the result in Example 4 above.

That is, as a result of measuring the prepared compound through XRPD, it was confirmed that the same crystal form as in Example 4 was obtained.

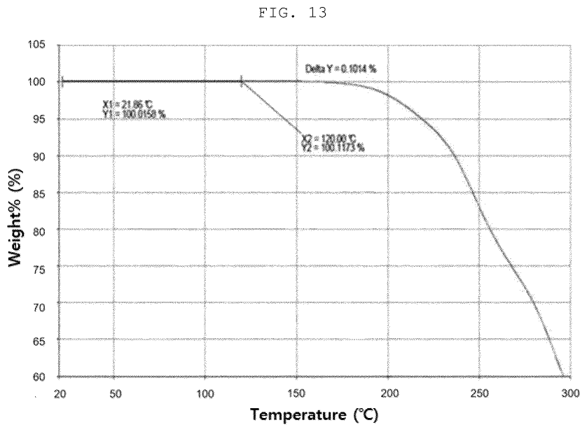

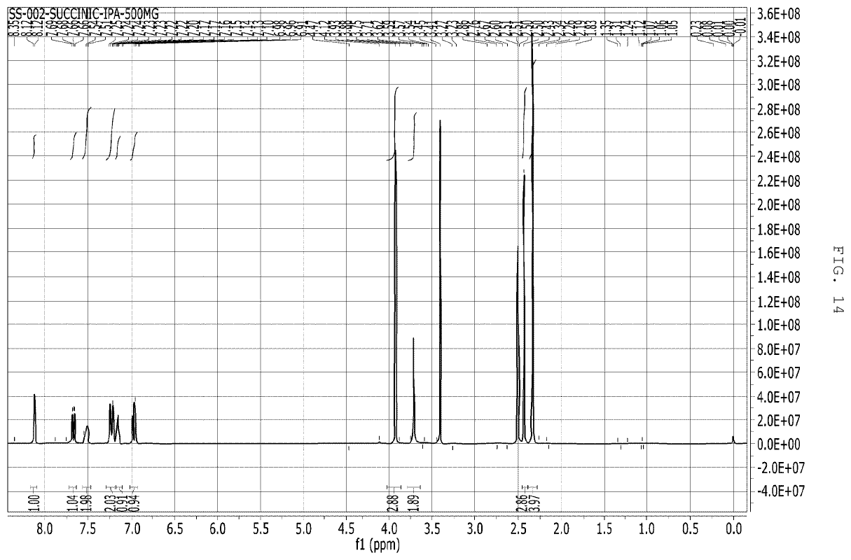

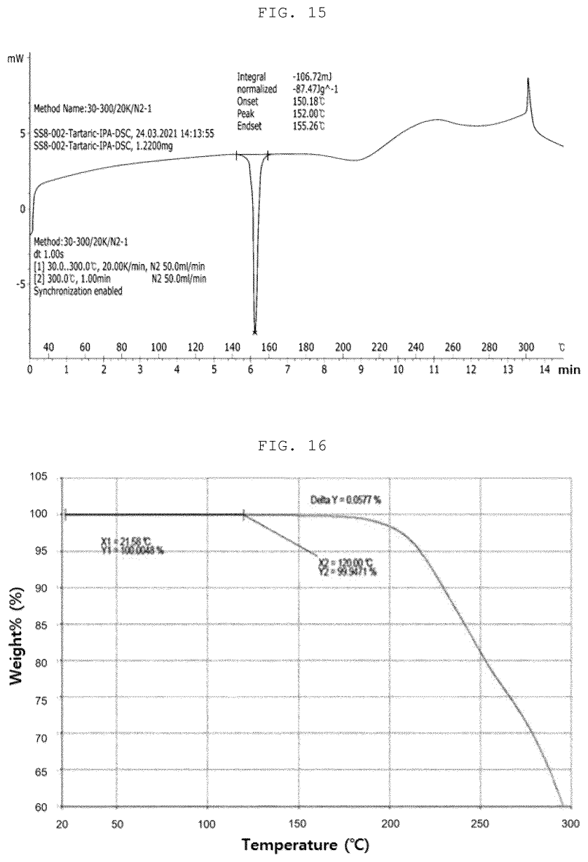

The results of DSC, TGA, and ¹H-NMR analysis were shown in FIGS. 12, 13 and 14, respectively.

As can be confirmed through the experiment, it was confirmed that the melting point was 141.04° C. and the TGA was about 0.10% to secure increased stability of materials and ease of storage. In other words, through the results, the production of the crystalline form was confirmed and it was confirmed that not only the content of residual solvent was low, but also a melting point (MP) value suitable for commercial use was measured.

### <Example 7> Preparation of L-tartrate salt of 1-(5-(2-fluorophenyl)-4-methoxy-1-((6-methoxypyridin-3-yl) sulfonyl)-1H-pyrrole-3-yl)-N-methylmethanamine

50.18 mg of 1-(5-(2-fluorophenyl)-4-methoxy-1-((6-methoxypyridin-3-yl)sulfonyl)-1H-pyrrole-3-yl)-N-methylmethanamine free base was weighed and put into a glass vial, and then dissolved in 0.6 mL of isopropyl alcohol at 25° C. Then, 64.74 μL of L-tartaric acid (2 M in H₂O) was added to the vial. The sample was stirred continuously for 24 hours in a magnetic stirrer under room temperature conditions, and after stirring for 24 hours, a solid precipitate was separated by centrifugation. Next, the wet solid was further dried under low pressure conditions at room temperature, and the dried solid was analyzed.

The resulting salt was analyzed using XRPD, DSC, TGA and ¹H-NMR.

A brief summary of the results of the analysis above was shown in Table 11 below.

TABLE 11

| | | TGA weight | ¹H-NMR (Ratio of |
|---|---|---|---|
| XRPD | DSC | loss (% w/w) | cation and anion) |
| Crystalline | Melting onset: | Weight | 1:1 |
| Form | 150.18° C. | loss: ~0.06%, | |
| | Endset: 155.26° C. | 120° C. | |
| | Peak: 152.00° C. | | |
| | (87.47 J/g) | | |

As a result of XRPD analysis, it was confirmed that one crystalline form was produced.

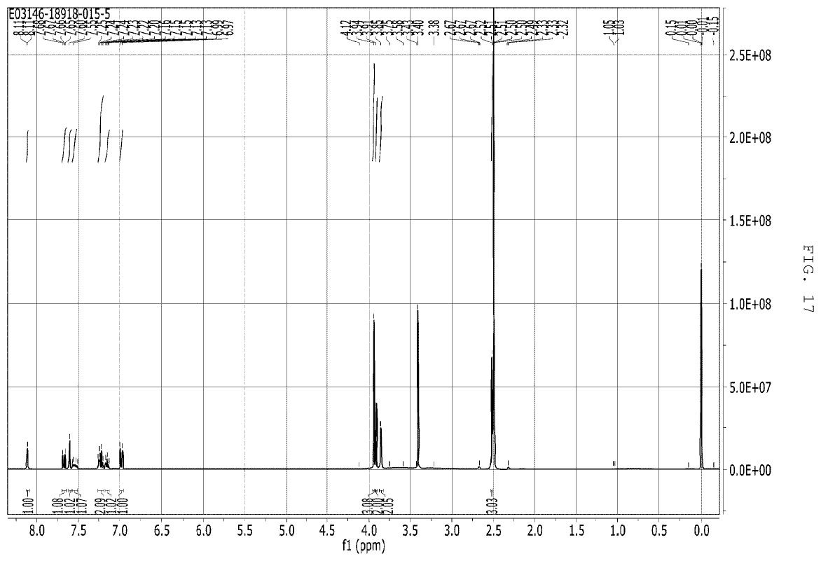

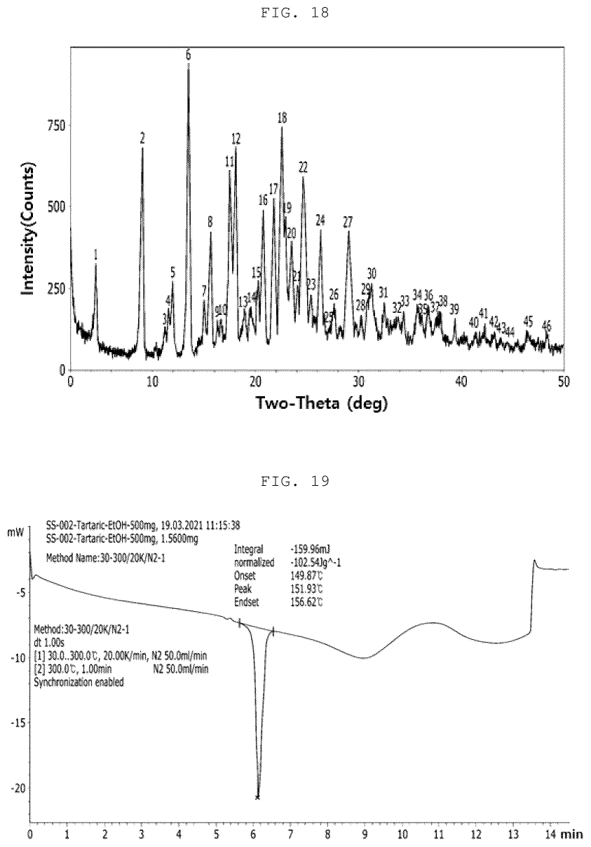

The results of DSC, TGA, and ¹H-NMR analysis were shown in FIGS. 15, 16 and 17, respectively.

As can be confirmed through the experiment, it was confirmed that the melting point was 152.00° C. and the TGA was about 0.06% to secure increased stability of materials and ease of storage.

### <Example 8> Preparation of L-tartrate salt of 1-(5-(2-fluorophenyl)-4-methoxy-1-((6-methoxypyridin-3-yl) sulfonyl)-1H-pyrrole-3-yl)-N-methylmethanamine

500.16 mg of 1-(5-(2-fluorophenyl)-4-methoxy-1-((6-methoxypyridin-3-yl)sulfonyl)-1H-pyrrole-3-yl)-N-methylmethanamine free base was weighed and put into a glass vial, and then dissolved in 2 mL of ethanol at 25° C. Then, 647.44 μL (2 M) of L-tartaric acid was added to the vial. The sample was stirred continuously for 24 hours in a magnetic stirrer under room temperature conditions, and after stirring for 24 hours, a solid precipitate was separated by centrifugation. Then, the wet solid was dried at 40° C. for 20 hours, and then a light gray dried powder was obtained.

The resulting salt was analyzed using XRPD, DSC, TGA and ¹H-NMR.

A brief summary of the results of the analysis above was shown in Table 12 below.

TABLE 12

| | | TGA weight | ¹H-NMR (Ratio of |
|---|---|---|---|
| XRPD | DSC | loss (% w/w) | cation and anion) |
| Crystalline | Melting onset: | Weight | 1:1 |
| Form | 149.87° C. | loss: ~0.03%, | |
| | Endset: 156.62° C. | 120° C. | |
| | Peak: 151.93° C. | | |
| | (102.54 J/g) | | |

The XRPD analysis results were shown in FIG. 18.

As a result of measuring XRPD of the prepared compounds, the values of FIG. 18 were confirmed. Specifically, XRPD patterns with peaks were shown at 8.99, 13.52, 15.64, 17.54, 18.07, 20.74, 21.79, 22.57, 22.88, 24.68, 26.32, and 29.03° 2θ±0.2°. In particular, characteristic peaks were identified at 8.99, 13.52, 17.54, 18.07, 20.74, 21.79, 22.57, and 24.68° 2θ±0.2°.

That is, as a result of measuring the prepared compound through XRPD, it was confirmed that the same crystal form as in Example 7 was obtained.

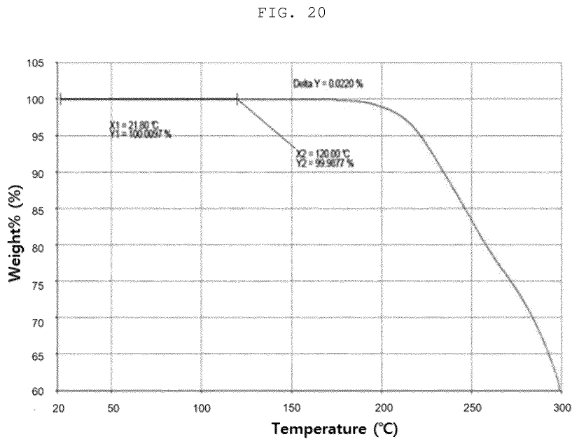

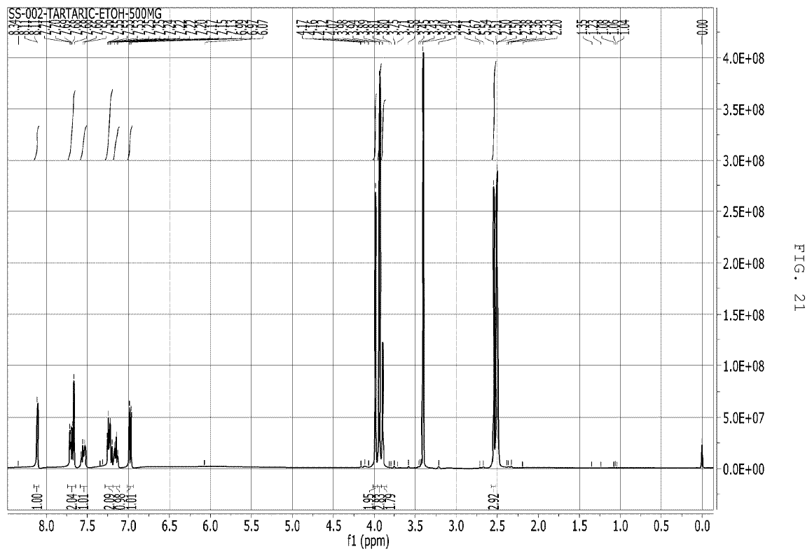

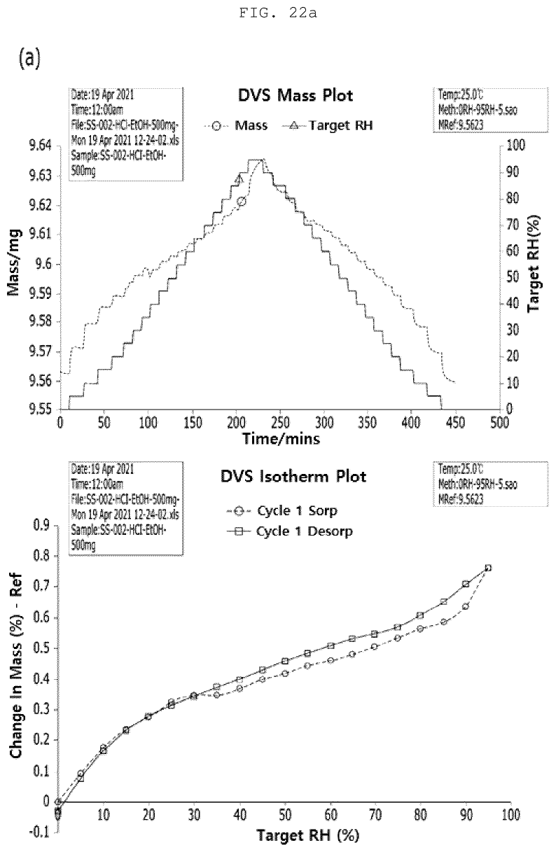

The results of DSC, TGA, and ¹H-NMR analysis were shown in FIGS. 19, 20 and 21, respectively.

It was confirmed that the melting point was 151.93° C. and the TGA was about 0.03% to secure increased stability of materials and ease of storage.

### <Experimental Example 1> Measurement of Hygroscopicity

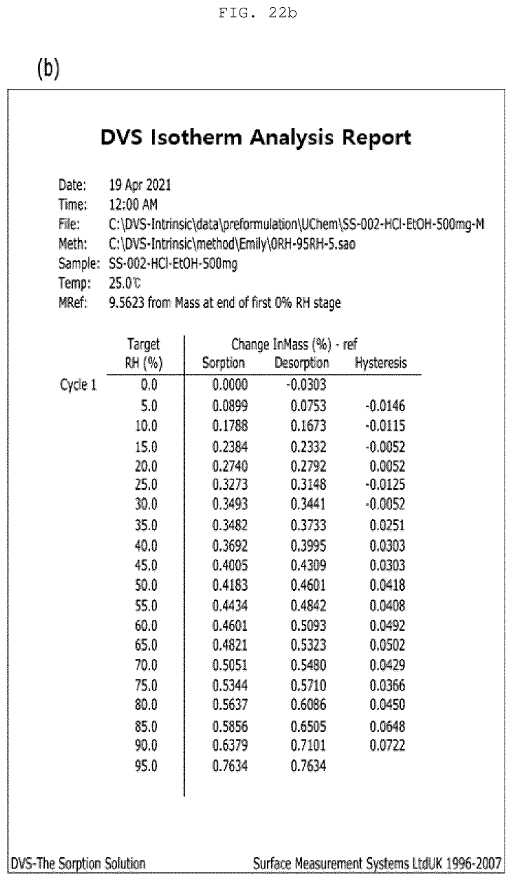

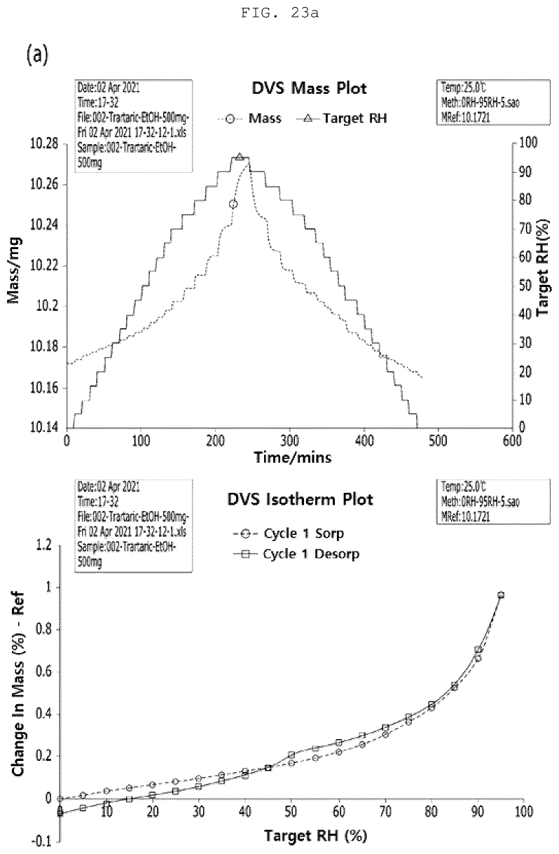

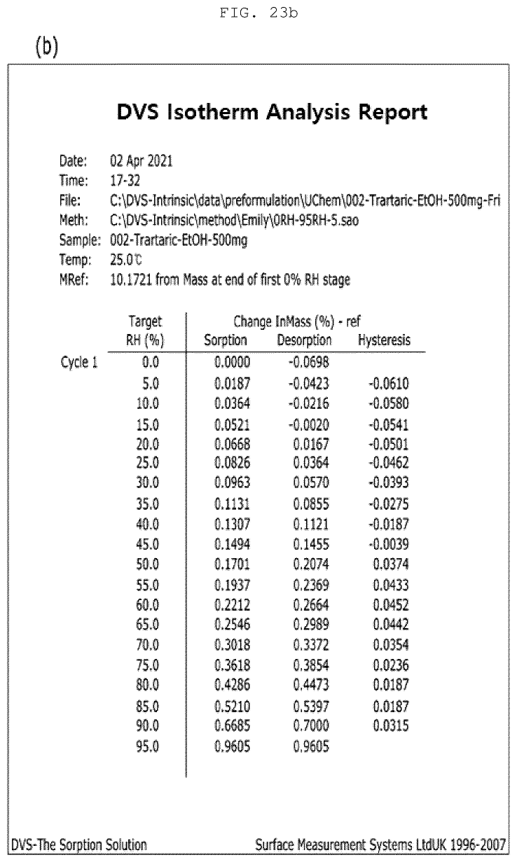

Using the dynamic vapor sorption (DVS) test method described above, the hygroscopicity was measured for the hydrochloride salt prepared in Example 1 and the L-tartrate salt prepared in Example 8, and the results thereof were shown in FIGS. 22 and 23.

As can be seen from the drawings, both hydrochloride salt and L-tartrate salt in the present disclosure showed very low hygroscopicity.

Specifically, when measuring the hygroscopicity of the hydrochloride salt of Example 1 and the L-tartrate salt of Example 8 through DVS at 25° C. and 0 to 95% humidity, very low hygroscopicity was shown by a change in weight of 0.2% or less.

### <Experimental Example 2> Evaluation of Solubility in Biorelevant Media

Solubility in biorelevant media, such as SGF, FeSSIF, and FaSSIF was confirmed with respect to the free base and the hydrochloride salt, succinate salt and L-tartrate salt of 1-(5-(2-fluorophenyl)-4-methoxy-1-((6-methoxypyridin-3-yl)sulfonyl)-1H-pyrrole-3-yl)-N-methylmethanamine that was confirmed through the salt screening analysis result.

Buffers for the biorelevant media were prepared as follows.

Water: Laboratory Milli-Q purified water.

SGF (Stimulated Gastric Fluid): 2.0 g of sodium chloride and 7 mL of hydrochloric acid dissolved in 1000 mL of water were used.

FaSSIF (Fasted State Simulated Intestinal Fluid): A commercial product purchased from Bio-Relevant Company and manufactured according to instructions was used.

FeSSIF (Fed State Simulated Intestinal Fluid): A commercial product purchased from Bio-Relevant Company and prepared according to instructions was used.

For the experiment, about 10 mg of a compound was weighed into each glass vial and then added with 1 mL of a medium (final concentration of 10 mg/mL). Then, the sample was continuously stirred in a magnetic stirrer at 37° C. at a speed of 200 rpm. After stirring for 1 hour and 24 hours, 0.5 mL of the sample solution was transferred to a 1.5 mL centrifuge tube and centrifuged at 12,000 rpm for 5 minutes. The supernatant was diluted with suitable methanol and analyzed by HPLC.

The free acid was selected as a standard sample to quantify the solubility, and about 10 mg of the free acid was weighed into a 25 mL flask and dissolved in methanol to the curve.

The residue of the compound in water was tested by XRPD to determine the solid state.

The experimental results were shown in Table 13.

TABLE 13

| | | Solubility-1 h | Solubility-24 h | |
|---|---|---|---|---|
| | | Concentration | Concentration | T |
| Compound | Media | (mg/mL) | (mg/mL) | (° C.) |
| Free base | water | 8.56 | 8.63 | 37 |
| | SGF | >10 | >10 | 37 |
| | FaSSIF | 1.44 | 1.44 | 37 |
| | FeSSIF | 4.57 | 4.77 | 37 |
| Hydrochloride | water | >10 | >10 | 37 |
| salt | SGF | >10 | >10 | 37 |
| | FaSSIF | 9.71 | 5.92 | 37 |
| | FeSSIF | 4.45 | 4.37 | 37 |
| Succinate | water | >10 | >10 | 37 |
| salt | SGF | >10 | >10 | 37 |
| | FaSSIF | 8.10 | >10 | 37 |
| | FeSSIF | 4.78 | 4.72 | 37 |
| L-tartrate | water | >10 | >10 | 37 |
| salt | SGF | >10 | >10 | 37 |
| | FaSSIF | 8.16 | >10 | 37 |
| | FeSSIF | 4.55 | 4.54 | 37 |

As can be confirmed above, the salt according to the present disclosure showed an overall improved effect in terms of solubility. In particular, basically, there was the possibility to show very rapid drug efficacy through an increase in water solubility, and very good solubility was exhibited under fasted state simulated intestinal fluid (FaSSIF) conditions that mimic the small intestine where most drug absorption occurs. This action shows that the salts according to the present disclosure are able to show high solubility regardless of meals, and to have excellent bioavailability, and the like, due to high absorption compared to the free base.

### <Experimental Example 3> Evaluation of Drug Stability

10 mg and 40 mg of the free base and the hydrochloride salt of 1-(5-(2-fluorophenyl)-4-methoxy-1-((6-methoxypyridin-3-yl)sulfonyl)-1H-pyrrole-3-yl)-N-methylmethanamine were weighed into each vial and stored under stress conditions to investigate stability.

The stress conditions are as follows:

High temperature open condition: 60° C.;

The bottle neck of the vial was wrapped with aluminum foil with pinholes to avoid contamination, and the samples were analyzed by XRPD and HPLC at the onset (day 0), week 1 and week 2.

The results thereof were shown in Table 14 below.

TABLE 14

| | [symbol] | | % | RT (min) | 6.797 | 7.423 | 7.563 | [symbol] .327 | 10.063 | 10.277 | 10.81 [symbol] |
|---|---|---|---|---|---|---|---|---|---|---|---|
| [symbol] | (XRPD) | Assay % | [symbol] | RRT | 0.68 | 0.74 | 0.75 | 0.93 | 1.00 | 1.02 | 1.07 |
| [symbol] | [symbol] | 100.00 | 0.85 | | | | | 0.1 [symbol] | 93.15 | | 0.66 |
| [symbol] | [symbol] | 87.44 | 20.94 | | 0.43 | [symbol] .86 | 1.00 | 0.1 [symbol] | 73.0 [symbol] | 9.95 | 0.57 |
| [symbol] | [symbol] | [symbol] .29 | 35.00 | | 0.3 [symbol] | [symbol] | [symbol] | 0.14 | 65.00 | 16. [symbol] | 0. [symbol] |
| [symbol] | Crystalline [symbol] | 100.50 | 0. [symbol] | | | | | | [symbol] | | 0.1 [symbol] |
| [symbol] | Crystalline [symbol] | 106.14 | 0.28 | | | | | | 9 [symbol] .72 | | 0. [symbol] |
| [symbol] | Crystalline [symbol] | 10 [symbol] .7 [symbol] | 0.19 | | | | | | [symbol] .81 | | 0. [symbol] |
| [symbol] | Crystalline | 100.00 | 0. [symbol] 1 | | | | | | 98. [symbol] | | 0.11 |
| [symbol] | Crystalline | [symbol] 9.60 | 0.12 | | | | | | 99. [symbol] | | 0.12 |
| [symbol] | Crystalline | 97.04 | 0.12 | | | | | | [symbol] | | 0.12 |
| [symbol] | Crystalline | 100.00 | 0.40 | | | | | | [symbol] | | 0.40 |
| [symbol] | Crystalline | 95.35 | 0.42 | | | | | | [symbol] | | 0.42 |
| [symbol] | Crystalline | 10 [symbol] .41 | 0.43 | | | | | | [symbol] .57 | | 0.43 |

[symbol]

indicates data missing or illegible when filed

As can be confirmed in Table 14, the hydrochloride salt according to the present disclosure showed excellent stability under harsh conditions. On the other hand, it was shown that the free base had a high impurity content under harsh conditions, which had a great limitation in commercial use.

1. A hydrochloride salt of 1-(5-(2-fluorophenyl)-4-methoxy-1-((6-methoxypyridin-3-yl)sulfonyl)-1H-pyrrole-3-yl)-N-methylmethanamine represented by the following Chemical Formula I:

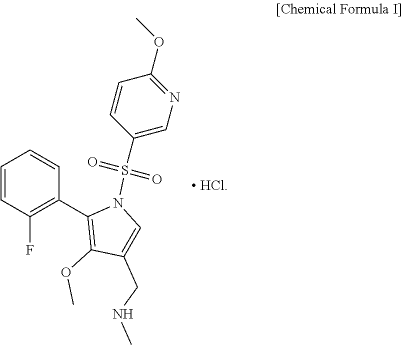

2. The hydrochloride salt of claim 1, wherein the hydrochloride salt is in a crystalline form.

3. The hydrochloride salt of claim 2, wherein the crystalline form is a crystalline form I and comprises, in an X-ray powder diffraction (XRPD) graph, at least three diffraction peaks at 2-theta (2θ) angle values selected from the group consisting of 16.53±0.2, 20.53±0.2, 21.32±0.2, 24.25±0.2, 26.78±0.2, 27.32±0.2 and 28.04±0.2.

4. The hydrochloride salt of claim 3, wherein the crystalline form I further comprises, in the X-ray powder diffraction (XRPD) graph, any one or more diffraction peaks at 2-theta (2θ) angle values selected from the group consisting of 8.97±0.2, 18.15±0.2, 25.45±0.2, and 32.40±0.2.

5. The hydrochloride salt of claim 2, wherein the crystalline form is a crystalline form II and comprises, in the X-ray powder diffraction (XRPD) graph, at least three diffraction peaks at 2-theta (2θ) angle values selected from the group consisting of 7.96±0.2, 13.22±0.2, 15.64±0.2, 16.04±0.2, 18.16±0.2, 22.88±0.2 and 25.18±0.2.

6. The hydrochloride salt of claim 5, wherein the crystalline form II further comprises, in the X-ray powder diffraction (XRPD) graph, any one or more diffraction peaks at 2-theta (2θ) angle values selected from the group consisting of 21.12±0.2, 24.12±0.2, 27.80±0.2, and 31.12±0.2.

7. The hydrochloride salt of claim 3, wherein the crystalline form has an endothermic transition peak value at 177 to 190° C. in a differential scanning calorimetry (DSC) graph.

8. The hydrochloride salt of claim 5, wherein the crystalline form has an endothermic transition peak value at 179 to 191° C. in a differential scanning calorimetry (DSC) graph.

9. The hydrochloride salt of claim 3, wherein the crystalline form has a thermogravimetric analysis (TGA) pattern showing a weight loss of less than 0.2 wt % at 120° C. or less.

10. The hydrochloride salt of claim 5, wherein the crystalline form has a thermogravimetric analysis (TGA) pattern showing a weight loss of less than 0.1 wt % at 120° C. or less.

11. A succinate salt of 1-(5-(2-fluorophenyl)-4-methoxy-1-((6-methoxypyridin-3-yl)sulfonyl)-1H-pyrrole-3-yl)-N-methylmethanamine represented by the following Chemical Formula II:

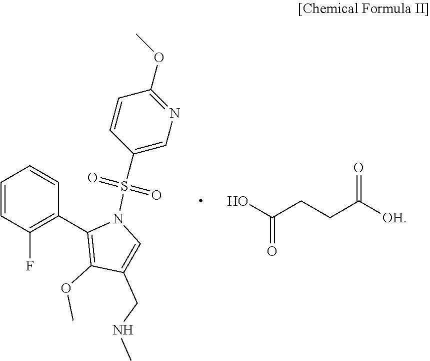 [SMILES: CNCc1cn(S(=O)(=O)c2ccc(OC)nc2)c(-c2ccccc2F)c1OC ; O=C(O)CCC(=O)O]

12. The succinate salt of claim 11, wherein the succinate salt is in a crystalline form.

13. The succinate salt of claim 12, wherein the crystalline form comprises, in an X-ray powder diffraction (XRPD) graph, at least three diffraction peaks at 2-theta (2θ) angle values selected from the group consisting of 17.14±0.2, 18.70±0.2, 19.74±0.2, 21.51±0.2, 22.75±0.2, 23.53±0.2, 25.81±0.2, and 28.40±0.2.

14. The succinate salt of claim 13, wherein the crystalline form further comprises, in the X-ray powder diffraction (XRPD) graph, any one or more diffraction peaks at 2-theta (2θ) angle values selected from the group consisting of 12.75±0.2, 14.09±0.2, 15.02±0.2, 20.42±0.2, and 29.82±0.2.

15. The succinate salt of claim 12, wherein the crystalline form has an endothermic transition peak value at 135 to 147° C. in a differential scanning calorimetry (DSC) graph.

16. The succinate salt of claim 15, wherein the crystalline form has the endothermic transition peak value at 141±2° C. in the differential scanning calorimetry (DSC) graph.

17. The succinate salt of claim 12, wherein the crystalline form has a thermogravimetric analysis (TGA) pattern showing a weight loss of less than 0.2 wt % at 120° C. or less.

18. An L-tartrate salt of 1-(5-(2-fluorophenyl)-4-methoxy-1-((6-methoxypyridin-3-yl)sulfonyl)-1H-pyrrole-3-yl)-N-methylmethanamine represented by the following Chemical Formula III:

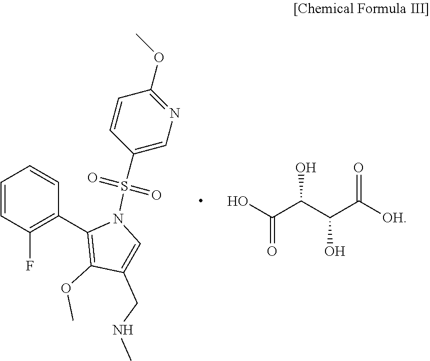 [SMILES: CNCc1cn(S(=O)(=O)c2ccc(OC)nc2)c(-c2ccccc2F)c1OC ; O=C(O)[C@H](O)[C@@H](O)C(=O)O]

19.-24. (canceled)

25. A method preventing or treating gastrointestinal ulcers, gastrointestinal inflammatory diseases or gastric acid-related diseases in a subject, the method comprising administering to the subject a pharmaceutical composition comprising the hydrochloride salt according to claim 1.

26. The method of claim 25, wherein the gastrointestinal ulcer, gastrointestinal inflammatory disease or gastric acid-related disease is any one or more selected from the group consisting of peptic ulcer, gastric ulcer, duodenal ulcer, NSAID-induced ulcer, acute stress ulcer, Zollinger-Ellison syndrome, Helicobacter pylori infection, gastritis, erosive esophagitis, non-erosive esophagitis, reflux esophagitis, inflammatory bowel disease, symptomatic gastroesophageal reflux disease (symptomatic GERD), functional dyspepsia, gastric cancer, gastric MALT lymphoma, hyperacidity, and upper gastrointestinal bleeding due to invasive stress.
